SEQUENCE LISTING (36 sequences)

SEQ ID NO: 1 - protein, 10 aa, Homo sapiens
  MOD_RES (1)..(1): Pyroglutamic acid or 5-oxoproline
EHWSYGLRPG

SEQ ID NO: 2 - protein, 16 aa, Artificial Sequence
  PEPTIDE (1)..(16): Amino acid sequence 829-844 of the Tetanus Toxoid Precursor (Tentoxylysin)
MQYIKANSKFIGITEL

SEQ ID NO: 3 - protein, 20 aa, Artificial Sequence
  PEPTIDE (1)..(20): Amino acid sequence 378-398 of the Plamodium falciparum circumsporozoite protein
DEKKIAKMEKASSVFNVVNS

SEQ ID NO: 4 - protein, 21 aa, Artificial Sequence
  PEPTIDE (1)..(21): Amino acid sequence 947-967 of Tetanus Toxoid Precursor (Tentoxylysin)
FNNFTVSFWLRVPKVSASHLE

SEQ ID NO: 5 - protein, 4 aa, Artificial Sequence
  Synthetic peptide
GPSL

SEQ ID NO: 6 - protein, 6 aa, Artificial Sequence
  Synthetic peptide
SSGPSL

SEQ ID NO: 7 - protein, 8 aa, Artificial Sequence
  Synthetic peptide
SSGPSLKL

SEQ ID NO: 8 - protein, 16 aa, Artificial Sequence
  PEPTIDE (1)..(16): Amino acid sequence 288-302 of the measles virus fusion protein, F
LSEIKGVIVHRLEGVE

SEQ ID NO: 9 - protein, 31 aa, Artificial Sequence
  Chimeric peptide made up of amino acid sequence 288-302 of the Measels virus fusion protein, F linked by a spacer peptide to amino acid sequence 2-10 of the GnRH hormone
KLLSEIKGVIVHRLEGVEGPSLHWSYGLRPG

SEQ ID NO: 10 - protein, 34 aa, Artificial Sequence
  MOD_RES (1)..(1): Amidated phenylalanine
FNNFTVSFWLRVPKVSASHLEGPSLHWSYGLRPG

SEQ ID NO: 11 - protein, 28 aa, Artificial sequence
  Chimeric peptide consisting of amino acid sequence 830-844 of the Tetanus toxoid precursor (Tentoxylysin) linked by a spacer to amino acid sequence 2-10 of the GnRH hormone
QYIKANSKFIGITELGPSLHWSYGLRPG

SEQ ID NO: 12 - protein, 33 aa, Artificial Sequence
  Chimeric peptide consisting of amino acid sequence 378-398 of the Plasmodium falciparum CSP protein linked by a spacer to amino acid sequence 2-10 of the GnRH hormone
DEKKIAKMEKASSVFNVVNSGPSLHWSYGLRPG

SEQ ID NO: 13 - protein, 34 aa, Artificial Sequence
  Chimeric peptide consisting of amino acid sequence 1-10 of the GnRH hormone linked by a spacer to amino acid sequence 288-302 of the Measles virus fusion protein,
EHWSYGLRPGSSGPSLKLLSEIKGVIVHRLEGVE

SEQ ID NO: 14 - protein, 37 aa, Artificial Sequence
  Chimeric peptide consisting of amino acid sequence 1-10 of the  GnRH hormone linked by a spacer to amino acid sequence 947-967 of the Tetanus toxoid precursor (Tentoxylysin)
EHWSYGLRPGSSGPSLFNNFTVSFWLRVPKVSASHLE

SEQ ID NO: 15 - protein, 31 aa, Artificial
  Chimeric peptide consisting of amino acid sequence 1-10 of the GnRH hormone linked by a spacer to amino sequence 830-844 of the Tetanus toxoid precursor (Tentoxylysin)
EHWSYGLRPGSSGPSLQYIKANSKFIGITEL

SEQ ID NO: 16 - protein, 36 aa, Artificial Sequence
  Chimeric peptide consisting of amino acid sequence 1-10 of the GnRH hormone linked by a spacer to amino acid sequence 378-398 of the Plasmodium falciparum circumsporozoite (CSP) protein
EHWSYGLRPGSSGPSLDEKKIAKMEKASSVFNVVNS

SEQ ID NO: 17 - protein, 47 aa, Artificial Sequence
  Chimeric peptide consisting of amino acid sequence 1-10 of the GnRH hormone linked by a spacer to amino acid sequence 288-302 of the Measles virus protein F linked by a spacer to amino acid sequence 2-10 of the GnRH hormone
EHWSYGLRPGSSGPSLKLLSEIKGVIVHRLEGVEGPSLHWSYGLRPG

SEQ ID NO: 18 - protein, 50 aa, Artificial Sequence
  Chimeric peptide consisting of amino acid sequence 1-10 of human GnRH linked by a spacer to amino acid sequence 947-967 of the Tetanus toxoid precursor (Tentoxylysin) protein linked by a spacer to amino acid sequence 2-10 of human GnRH
EHWSYGLRPGSSGPSLFNNFTVSFWLRVPKVSASHLEGPSLHWSYGLRPG

SEQ ID NO: 19 - protein, 46 aa, Artificial Sequence
  Chimeric peptide consisting of amino acid sequence 1-10 of human GnRH linked by a spacer to amino acid sequence 830-844 of Tetanus toxoid precursor (Tentoxylysin) linked by a spacer to amino acid sequence 1-10 of GnRH
EHWSYGLRPGSSGPSLQYIKANSKFIGITELSSGPSLHWSYGLRPG

SEQ ID NO: 20 - protein, 51 aa, Artificial Sequence
  Chimeric peptide consisting of amino acid sequence 1-10 of human GnRH linked by a spacer to amino acid sequence 378-398 of Plasmodium falciparum circumsporozoite (CSP) protein
EHWSYGLRPGSSGPSLDEKKIAKMEKASSVFNVVNSSSGPSLHWSYGLRPG

SEQ ID NO: 21 - protein, 20 aa, Hepatitis B virus
MDIDPYKEFGATVELLSFLP

SEQ ID NO: 22 - protein, 20 aa, Hepatitis B virus
PHHYALRQAILCWGELMYLA

SEQ ID NO: 23 - protein, 20 aa, Hepatitis B virus
LLWFHISCLTFGRETVIEYL

SEQ ID NO: 24 - protein, 15 aa, Hepatitis B virus
EYLVSFGVWIRTPPA

SEQ ID NO: 25 - protein, 20 aa, Hepatitis B virus
VSFGVWIRTPPAYRPPNAPI

SEQ ID NO: 26 - protein, 10 aa, Hepatitis C virus
ADLMGYIPLV

SEQ ID NO: 27 - protein, 10 aa, Hepatitis C virus
LLALLSCLTV

SEQ ID NO: 28 - protein, 9 aa, Hepatitis C virus
LLCPAGHAV

SEQ ID NO: 29 - protein, 10 aa, Hepatitis C virus
KLVALGINAV

SEQ ID NO: 30 - protein, 9 aa, Hepatitis C virus
SLMAFTAAV

SEQ ID NO: 31 - protein, 10 aa, Hepatitis C virus
LLFNILGGWV

SEQ ID NO: 32 - protein, 9 aa, Hepatitis C virus
ILDSFDPLV

SEQ ID NO: 33 - protein, 33 aa, Artificial sequence
  Chimeric peptide consisting of (amino acids 1-20) tetanus toxin Th epitope 10 sequence linked by a spacer peptide GPSL (amino acids 21-24) to amino acid sequence 2-10 of human GnRH (amino acids 25-33)
LRVDNKNYFPCRDGFGSIMQGPSLHWSYGLRPG

SEQ ID NO: 34 - protein, 33 aa, Artificial sequence
  Chimeric peptide consisting of (amino acids 1-20) tetanus toxin Th epitope 11 sequence linked by a spacer peptide GPSL (amino acids 21-24) to amino acid sequence 2-10 of human GnRH (amino acids 25-33)
NEIVSYNTKNKPLNFNYSLDGPSLHWSYGLRPG

SEQ ID NO: 35 - protein, 33 aa, Artificial sequence
  Chimeric peptide consisting of (amino acids 1-20) Diphtheria toxin Th epitope 2 sequence linked by a spacer peptide GPSL (amino acids 21-24) to amino acid sequence 2-10 of human GnRH (amino acids 25-33)
QSIALSSLMVAQAIPLVGELGPSLHWSYGLRPG

SEQ ID NO: 36 - protein, 33 aa, Artificial sequence
  Chimeric peptide consisting of (amino acids 1-20) tetanus toxin Th epitope 5 sequence linked by a spacer peptide GPSL (amino acids 21-24) to amino acid sequence 2-10 of human GnRH (amino acids 25-33)
GVLLPTIPGKLDVNKSKTHIGPSLHWSYGLRPG

(19) United States
(12) Patent Application Publication
(10) Pub. No.: US 2005/0106137 A1
(43) Pub. Date: May 19, 2005

(54) Chimeric peptide immunogens

(76) Inventor: Stephen Grimes, Davis, CA (US)

Correspondence Address: WHITE & CASE LLP;PATENT DEPARTMENT, 1155 AVENUE OF THE AMERICAS, NEW YORK, NY, 10036, US

(21) Appl. No.: 10/931,566

(22) Filed: Aug. 31, 2004

Related U.S. Application Data
(63) Continuation-in-part of application No. 09/848,834, filed on May 4, 2001, now Pat. No. 6,783,761.
(60) Provisional application No. 60/202,328, filed on May 5, 2000.

(51) Int. Cl. A61K 39/395; C07K 16/44

(52) U.S. Cl. 424/133.1; 530/387.3

(57) ABSTRACT

Chimeric peptide epitopes can serve as effective immunogens against hormones and other small peptides or proteins. Immunogenic peptides are selected from promiscuous helper T-lymphocyte epitopes and synthesized together with self antigenic peptide sequences optionally fused through a spacer moiety. Examples of the chimeric peptide immunogens of the invention include peptide sequences which may be from any antigen, such as a gonadotropin releasing hormone (GnRH), linked with an immunogenic peptide sequence such as a promiscuous helper T-lymphocyte epitope of measles virus protein F, tetanus toxoid, or malaria protein CSP. Compositions of the chimeric immunogen are found effective in eliciting high and specific anti-GnRH antibody titers. This invention also relates to compositions and methods for synergistically enhancing or suppressing an immune response to a target antigen.

This application is a continuation-in-part of U.S. Ser. No. 09/848,834 filed May 4, 2001, now issued as U.S. Pat. No. 6,783,761, which claims priority from provisional application No. 60/202,328, filed May 5, 2000. This application also claims the benefit of U.S. Ser. No. 10/866,038 and U.S. Ser. No. 10/866,469 both filed Jul. 6, 204 as divisional applications of U.S. Ser. No. 09/848,834 listed above. The specifications of these applications are hereby incorporated by reference in their entireties.

### FIELD OF THE INVENTION

The invention is related to chimeric peptides having immunogenic efficacy, comprising a hormone epitope and promiscuous helper T-cell epitope for the production of high titers of anti-hormone antibodies. The invention is also related to synergystic or suppressive interaction of chimeric immunogens administered to provide synergy or suppression of an antibody response to a target antigen.

### BACKGROUND OF THE INVENTION

The success of an antigenic composition is linked to its immunogenicity, that is, the ability to produce a sufficiently high titer of antibodies to react or bind with the target antigen or so as to neutralize its effects. The immunogenicity depends on the effectiveness by which the antigen causes the body's immune system to mount a response which can be generally assessed on the basis of the antibody titer in the blood of the immunized animal such as a mammal, including a human.

Antigenic formulations can be prepared for antigens of low immunogenicity with constructs or mixtures of an immunomimic epitope of the target antigen and an immunogen not related to the target antigen so as to generate a strong immune response against the entire immunogenic construct or mixture so as to be effective against the specific target antigen.

In order to enhance or potentiate the immune defense system, so-called adjuvants in the form of oily substances and other potentiating and emulsifying agents are added to the antigenic formulations. In general, the adjuvant is mixed into the immunogenic emulsion formulation and simultaneously delivered with the antigen in the same administration, e.g., by injection. Specifically, antigenic formulations have been enhanced to target less immunogenic microorganisms or viral pathogens by the addition of so-called adjuvants comprising immune response-stimulating killed microbial cells, particles or fragments thereof. Moreover, immunogenic compositions may contain carrier components, including emulsions, liposomes, microparticles and implantable vehicles which may be metabolizable.

Immunization technology has been applied as a biological modifying means to immunize against various soluble and insoluble animal or human self-antigens, which are not normally recognized by the individual host's own immune defense, but which may be rendered immunogenic so as to stimulate or potentiate the individual's own immune response system. The self-antigens may include the surfaces of certain cells which are malfunctioning or malignant, and small proteins, enzymes or intercellular signals, such as, e.g., hormones or other factors, and/or their cognate receptors, whether normal or deficient. The lack of immunogenicity of these self-antigens has been often overcome by complexing or linking the non-immunogenic self-antigens with a pharmaceutically acceptable, i.e. non-toxic, immunogenic carrier so as to produce antibodies capable of binding, thereby neutralizing, the self-antigen of the subject animal or human patient.

The immunological methods can be used for example in the therapeutical hormone control or regulation and the treatment of patients afflicted with a disorder or disease.

Some immunogens suitable for hormone-regulation comprise hormone immunomimicking molecular moieties, which are conjugated or fused to immunogenic carriers, such as, e.g., proteins, or peptides or complex polysugars. The immunogenic constructs are usually administered as either an oil-in-water or a water-in-oil emulsion, containing an adjuvant capable of stimulating or potentiating an immune response.

An immune response is typically measured in terms of the production of specific anti-hormone antibodies. The hormones and cognate receptors which are targeted for control by the immunological methods are directly neutralized or inhibited by the antigen-binding reaction of circulating hormone specific antibodies elicited by the injected immunogenic constructs.

For example, an anti-hormone immunogen has been constructed to affect the regulation of the gonadotropin releasing hormone (see co-assigned U.S. Pat. No. 5,688,506). The Gonadotropin Releasing Hormone (abbreviated “GnRH”, also known as Luteinizing Hormone Releasing Hormone, abbreviated “LHRH”), is of central importance to the regulation of fertility. Johnson M et al., Essential Reproduction, 3^{rd} Edn. Blackwell Scientific Publications (1988). In both males and females, GnRH is released from the hypothalamus into the bloodstream and is transported through the bloodstream to the pituitary, where it induces the release of gonadotropins, luteinizing hormone (LH) and follicle stimulating hormone (FSH), by the gonadotrophs. These gonadotropins, in turn, act upon the gonads, inducing steroidogenesis and gametogenesis. Steroids released from the gonads into the circulation subsequently act upon various tissues. This gonadotropin related hormonal cascade can be halted by the neutralization of the biological activity of GnRH. Fraser H. M., Physiological Effects of Antibody to Lutenizing Hormone Releasing Hormone, Physiological Effects of Immunity Against Reproductive Hormones, Edwards and Johnson, Eds. Cambridge University Press (1976). As a consequence of GnRH neutralization, the gonadotropins and gonadal steroids are not released into the blood, and their biological activities are curtailed or eliminated by the direct and indirect action of specific anti-GnRH antibodies. By eliminating the physiological activity of GnRH, the cascade of hormonal regulation of fertility is interrupted and gametogenesis ceases. Consequently, GnRH neutralization halts the production of gametes. Thus, GnRH neutralization is an effective means of contraception.

A number of important diseases are affected by gonadotropins and particularly gonadal steroid hormones. Such diseases include breast cancer, uterine and other gynecological cancers, endometriosis, uterine fibroids, benign prostatic hypertrophy and prostate cancer, among others. Removal of the gonadal steroid hormonal stimuli for these diseases constitutes an important means of therapy. An effective method of accomplishing this is by immunologically neutralizing GnRH, to thereby eliminate or inhibit production of GnRH dependent gonadal steroids that induce and stimulate these diseases. McLachlan R. I. et al. Clinical Aspects of LHRH Analogues in Gynaecology: a Review, British Journal of Obstetrics and Gynaecology, 93:431-454 (1986); Conn P. M. et al. Gonadotropin-Releasing Hormone and Its Analogs, New England Journal of Medicine. 324:93-103 (1991) and Filicori M. GnRH Agonists and Antagonists, Current Clinical Status. Drugs. 35:63-82 (1988).

Since GnRH has the same amino acid sequence in all mammals (pGlu-His-Trp-Ser-Tyr-Gly-Leu-Arg-Pro-GlyNH₂, SEQ ID NO: 1 in the Sequence Listing), it is presumed that a single immunogen would be effective in all mammalian species, including humans. An anti-GnRH immunogenic construct, comprising the GnRH immunomimic domain in the form of peptide analogues, may be linked or conjugated to a carrier protein which is effectively immunogenic, such as, e.g., diphtheria toxoid, tetanus toxoid, keyhole limpet hemocyanin, bovine serum albumin, Hemophilus, or Pertussis extracts or filamentous Amycolata extracts. Consequently, the immune response to the GnRH-vaccine will be mostly directed against the carrier protein and secondarily, the attached hormone epitope moiety. In general, as an alternative approach, the immunogenicity of the immunomimic peptide can be enhanced by chemical modification with diazosulfuric acid groups.

Various anti-GnRH immunogenic compositions have been useful for producing specific anti-GnRH antibodies. Immunogenic conjugates of GnRH-immunomimic epitope peptide and immunogenic protein carriers have been used for immunization of vertebrate subjects against the hormone, GnRH (U.S. Pat. No. 5,688,506).

As another example, anti-hormone immunogens have been constructed, to affect or inhibit the activity of the stomach hormone gastrin, in particular, the major forms of gastrin, gastrin G17 and gastrin G34 (see U.S. Pat. Nos. 5,023,077, and 5,468,494). It has been found that especially G17 is involved in gastrointestinal disorders and diseases such as gastroesophageal reflux disease, gastric and duodenal ulceration and cancer.

However, it has been found that perhaps due to the comparatively huge size of the attached immunogenic carrier proteins, the immunization of the conjugate can induce anti-epitope specific suppression of the antibody (Sad et al. Immunology, 1985, 74:559; Schutze et al. J. Immunol, 1985, 135:231). Therefore, much smaller immunogenic proteins have been tried. Accordingly, short synthetic T-helper epitopes have been introduced to replace the large carrier molecules in conjugates to improve the efficacy of the anti-hormone or self antigenic immunogen. Sad et al. (Vaccine 1993, 11:1145-1149) synthesized peptides from DT and universal or highly promiscuous T-helper epitopes from TT (829-844 amino acids, SEQ ID NO: 2) or CSP (378-398 aa; SEQ ID NO: 3) in order to try to minimize genetic restriction of the immune response. To be effective, the GnRH vaccines of Sad et al. required Freund's Complete Adjuvant.

Gosh et al. (Int. Immunology, 1999, 11:1103-1110) reported that some synthetic LHRH (GnRH) chimeric vaccines elicited an immune response for sterilization of mice. However, the promiscuous helper T-cell (Th)-epitope candidate T1 (TT sequence 947-967 aa, SEQ ID NO: 4) was not regarded promiscuous enough to be applicable for a large number of animal species. It was also reported that in a shift, antisera from second bleedings reacted significantly with the anti-Th epitope (T2) and much less with the LHRH antigen.

U.S. Pat. No. 5,759,551, issued to Ladd et al., also reported chimeric antigens directed to LHRH. U.S. Pat. No. 6,559,282, issued to Wang, described vaccines comprising mixtures of chimeric LHRH peptide immunogens. However, these mixtures were used to address genetic restriction in an immunized population. The mixture being administered to improve the chance that one of chimeras in the mixture would elicit an antibody response sufficient to provide immunoefficacy in any particular individual in the population.

EP 1 117 421 B 1 issued to Stenaa, discloses the preparation of immunogenic, modified polypeptide antigens which are derived from weakly immunogenic antigens.

U.S. Pat. No. 5,662,909, issued to Becker et al., described simultaneous administration to an animal of different forms of the HA antigen of the influenza virus and the of OspA protein of the Borrelia burgdorferi spirochete, resulting in a synergistic immune response. However, these different forms were limited to different physio-chemical forms of the antigen.

These disclosures neither teach how to identify modulation of an antibody response elicited by a chimeric immunogen, nor do they show any evidence of synergy or suppression in the immunized individual.

### SUMMARY OF THE INVENTION

The present invention provides immunogens comprising a chimeric peptide of a hormone-immunomimic peptide epitope fused in sequence with an immunogenic epitope. The hormone immunogenic peptide can be fused either directly to or through a spacer sequence to an immunogenic peptide epitope.

These fusion peptides combine at least one epitope of a target substance which may be non-immunogenic in its natural state, with at least one immunogenic peptide sequence of suitable immunogenic proteins. The sequences of both target epitope and immunogen may be selected from the amino-terminal or carboxy-terminal region or both. A peptide also can be synthesized from the internal region of the peptide or protein. The fusion product may be acetylated at the amino-terminal end and amidated at the carboxy-terminal end of the peptide sequence. An embodiment of the invention provides a synthetic immunogenic fusion peptide selected from the group consisting of one or more than one peptide defined by SEQ ID NO: 10 and SEQ ID NO: 11.

One embodiment of the invention provides an anti-GnRH immunogen chimeric peptide construct comprising a suitable immunogenic epitope, such as, e.g., short peptide sequences selected from the measles virus protein F (MVF), tetanus toxoid (TT), or malaria plasmodium falciparum CSP protein. The invention also provides for methods of immunization with a composition comprising a chimeric peptide with one or more GnRH epitopes.

The invention also provides a composition that includes: a) a first chimeric immunogen which itself includes i) a helper T-lymphocyte epitope, fused through ii) a spacer moiety, to iii) an immunomimic of a target antigen; and b) a second chimeric immunogen comprising that includes i) a different helper T-lymphocyte epitope, fused through ii) a spacer moiety, to iii) the immunomimic of the target antigen; wherein the helper T-lymphocyte epitopes synergistically enhance or suppress the antibody response to the target antigen.

The present invention further provides a method of eliciting an antibody response to a target antigen in a patient, comprising: administering to the patient a composition that includes:
a) a first chimeric immunogen comprising i) a helper T-lymphocyte epitope, fused through ii) a spacer moiety, to iii) an immunomimic of the target antigen; and
b) a second chimeric immunogen comprising i) a different helper T-lymphocyte epitope, fused through ii) a spacer moiety, to iii) the immunomimic of the target antigen;
   wherein the helper T-lymphocyte epitopes synergistically enhance the antibody response to the target antigen.

The present invention yet further provides a method of suppressing an antibody response to a target antigen in a patient, the method includes administering to the patient a composition containing: a chimeric immunogen including i) a helper T-lymphocyte epitope that is not found in the target antigen, fused through ii) a spacer moiety, to iii) an immunomimic of the target antigen; wherein the helper T-lymphocyte epitope suppresses the antibody response to the target antigen.

The present invention also provides a pharmaceutical composition including two or more chimeric immunogens for modulating the production of antibodies against a target antigen; the composition includes
a) a first chimeric immunogen comprising i) a helper T-lymphocyte epitope, fused through ii) a spacer moiety, to iii) an immunomimic of the target antigen; and
b) a second chimeric immunogen comprising i) a different helper T-lymphocyte epitope, fused through ii) a spacer moiety, to iii) the immunomimic of the target antigen;
   wherein the helper T-lymphocyte epitopes synergistically enhance the antibody response to the target antigen.

### BRIEF DESCRIPTION OF THE DRAWINGS

FIG. 1 illustrates the mean Anti-GnRH antibody titers obtained from rabbits using chimeric anti-GnRH Immunogens A through J, and as controls, Immunogens K and L as well as conjugate immunogen C, GnRH:DT.

FIG. 2 illustrates the relationship between gross muscle reaction score and mean anti-GnRH Antibody Titer on GnRH Chimeras and Controls.

FIG. 3 illustrates the mean and median rabbit Anti-GnRH antibody titers induced by a mixture of GnRH chimeric peptides 2 and 3, in studies A and C.

FIG. 4 illustrates the mean rabbit Anti-GnRH Antibody Titers induced by chimeric immunogens in study B.

FIG. 5 illustrates the median rabbit Anti-GnRH Antibody Titers induced by chimeric immunogens in study B.

### DETAILED DESCRIPTION OF THE INVENTION

As used in the present specification, the terms “promiscuous helper T-cell epitope”, “helper T-lymphocyte epitope”, “T-lymphocyte epitope”, “T-helper epitope”, “Th epitope” and “T-cell epitope” are interchangeable, each for the other. The terms “spacer” and “linker” are also used interchangeably. Similarly, as used herein, the terms “chimeric peptide”, “fusion peptide”, “fusion protein”, and “chimera” are interchangeable. The terms “target antigen” and “target substance” are also used interchangeably.

Chimeric peptides comprising GnRH mimicking epitopes have been constructed and are useful in generating improved antibody titers.

Since self-antigen epitopes of gonadotropin releasing hormone (GnRH) are not inherently immunogenic the immune response may be aided by immunogenic constructs according to the invention wherein a target peptide epitope is located on the same synthesized peptide as is an immunogenic peptide epitope.

The present invention provides a composition that includes two or more chimeric immunogens for modulating, i.e. synergistically enhancing or suppressing the production of antibodies against a target antigen. Each of the chimeric immunogens of the mixture includes: i) a helper T-lymphocyte epitope, fused through ii) a spacer moiety, to iii) an identical immunomimic of the target antigen. The chimeric immunogens of the mixture each include a different helper T-lymphocyte epitope. The modulation of the immunogenicity of the component chimeric immunogens can be synergistic, or suppressive. That is, the mixture of chimeric immunogens displays either synergy, where the titer of antibodies elicited by the mixture is greater than the sum of the antibody titers elicited by each immunogen alone; or suppression, such that the mixture elicits a lower antibody titer than the sum of the titers of the individual chimeric immunogen components.

Alternatively, the chimeric immunogens of the invention can be administered singly to suppress an antibody response to a target antigen that is in the process of being elicited, or a pre-existing antibody response to a target antigen.

The chimeric immunogens of the invention can be synthetic, such as for instance, a synthetic peptide or a modified synthetic peptide produced by routine solid phase or liquid phase synthetic methods that are commercially available. Alternatively, the chimeric immunogens of the invention can be recombinant immunogens produced by recombinant DNA means from prokaryotic, or eukaryotic expression systems well known in the art. See, for instance, J. Sambrook & D. W. Russell, 2001 “Molecular Cloning, A Laboratory Manual” 3^{rd} Edn., Cold Spring Harbor Laboratory, Cold Spring Harbor, N.Y.

The chimeric immunogen compositions of the invention can be directed against any target antigen to which antibody response is desired to be elicited or suppressed. An immunomimic of this target antigen may be an actual fragment of the target antigen, such as an amino acid sequence fragment of the target antigen, or an immunomimic of the target antigen that elicits antibodies that bind the target antigen.

In one embodiment, the present invention provides chimeric immunogen compositions, wherein the target antigen is GnRH and the immunomimic of the GnRH target antigen can be any GnRH fragment of between about 5 amino acids up to the full length (10 amino acid) sequence of GnRH. In one particularly useful embodiment, the immunomimic of the GnRH target antigen is the contiguous amino acid sequence 2-10 of GnRH. In another particularly useful embodiment, the immunomimic of the GnRH target antigen is the full amino acid sequence 1-10 of GnRH.

The immunomimic of the GnRH target antigen can include an acetylated amino terminal glutamic acid, or an amidated carboxy-terminal glycine. Alternatively, in an embodiment where the GnRH immunomimic is attached to the T-lymphocyte epitope at a position other than the N-terminus or the C-terminus, the GnRH immunomimic can include both an acetylated amino terminal glutamic acid and an amidated carboxy-terminal glycine. In another alternative embodiment, the helper T-lymphocyte epitope can be fused through a spacer moiety to the amino terminus or the carboxy terminus of a GnRH immunomimic peptide.

Target antigens to which the chimeric immunogens of the invention can be directed include tumor antigens, graft antigens, viral antigens, bacterial antigens, other pathogen antigens, and artificial antigens. These target antigens also include non-peptide antigens such as bacterial capsular polysaccharides (e.g., the capsular Salmonella flagellin); non-natural peptides, such as for instance, poly-D-amino acids; and E. coli lipopolysaccharides as disclosed in U.S. Pat. Nos. 5,736,146 issued to Cohen et al.

Alternatively, the target antigen can be any peptide hormone. Peptide hormones include, for instance, somatostatin, amylin, angiotensin, bombesin, bradykinin, C5a anaphylatoxin, calcitonin, calcitonin-gene related peptide (CGRP), corticotropin releasing hormone (CRH), chemokines, cholecystokinin (CCK), endothelin, erythropoietin (EPO), follicle stimulating hormone (FSH), formyl-methionyl peptides, galanin, gastrin (including any of the gastrin hormone forms, such as gastrin-17, gastrin-34, glycine-extended gastrin-17, glycine-extended gastrin-34 and progastrin), gastrin releasing peptide, glucagon, glucagon-like peptide 1, glycoprotein hormones, gonadotropin, gonadotropin-releasing hormone, leptin, luteinizing hormone (LH), melanocortins, neuropeptide Y, neurotensin, oxytocin, parathyroid hormone, secretin, somatostatin, tachykinins, thyrotropin, thyrotropin releasing hormone, vasoactive intestinal polypeptide (VIP), vasopressin, and the like.

The helper T-lymphocyte epitope components of the chimeric immunogens useful in the practice of the present invention can be any helper T-lymphocyte epitope. For instance the T-lymphocyte epitope can be a T-lymphocyte epitope from the target antigen. Alternatively, the T-lymphocyte epitope can be from a different molecule, unrelated to the target antigen. For instance, T-lymphocyte epitopes useful in the practice of the present invention include the T-lymphocyte epitopes of Diphtheria toxoid (DT) and of Tetanus toxoid (TT). The T-lymphocyte epitopes of DT and of TT are used for illustration as model T-lymphocyte epitopes in of the present invention, however any of the many other available T-lymphocyte epitopes can be used.

Examples of T-lymphocyte epitopes that are particularly useful in the practice of the present invention include, for example, T-lymphocyte epitopes derived from viral antigens, bacterial antigens and antigens of other pathogens, as well as those derived from any of the other well known highly immunogenic molecules, such as, for instance keyhole limpet hemocyanin (KLH).

Helper T-lymphocyte epitopes particularly useful in the practice of the present invention include the hepatitis B virus (HBV) surface and core antigen helper T cell epitopes (HBsAg Th and HBc Th), the pertussis toxin helper T cell epitopes (PT Th), the Chlamydia trachomatis major outer membrane protein helper T cell epitopes (CT Th), the Schistosoma mansoni triose phosphate isomerase helper T cell epitopes (SM Th), and the Escherichia coli TraT helper T cell epitopes (TraT Th). The sequences of these pathogen-derived Th epitopes can be found in U.S. Pat. No. 5,759,551, issued to Ladd et al., as SEQ ID NOS: 2-9 and 42-52 therein. See also U.S. Pat. No. 6,559,282, issued to Wang at col. 3, line 63-col. 4, line 21. The helper T-lymphocyte epitopes of cholera toxin, Shigella and E. coli enterotoxin are also useful in the chimeric immunogens of the present invention.

Other specific examples of useful promiscuous helper T-lymphocyte epitopes include the T-helper epitopes from HBV, such as, for instance, HBc1-20, having the sequence: Met-Asp-Ile-Asp-Pro-Tyr-Lys-Glu-Phe-Gly-Ala-Thr-Val-Glu-Leu-Leu-Ser-Phe-Leu-Pro; and HBc50-69, having the sequence Pro-His-His-Tyr-Ala-Leu-Arg-Gln-Ala-Ile-Leu-Cys-Trp-Gly-Glu-Leu-Met-Tyr-Leu-Ala; and from the region of HBc100-139, including HBc100-119 having the sequence Leu-Leu-Trp-Phe-His-Ile-Ser-Cys-Leu-Thr-Phe-Gly-Arg-Glu-Thr-Val-Ile-Glu-Tyr-Leu (where Ile.sub. 116 is Leu in the HBV adw subtype), HBc117-131 having the sequence Glu-Tyr-Leu-Val-Ser-Phe-Gly-Val-Trp-Ile-Arg-Thr-Pro-Pro-Ala, and HBc120-139 having the sequence Val-Ser-Phe-Gly-Val-Trp-Ile-Arg-Thr-Pro-Pro-Ala-Tyr-Arg-Pro-Pro-Asn-Ala-Pr o-Ile. See U.S. Pat. No. 6,235,288, issued to Chisari, col. 11, line 62- col. 12, line 14.

Still other examples of useful promiscuous helper T-lymphocyte epitopes include sequences from the hepatitis C virus (HCV) genome, such as, for instance, sequences from the core protein (e.g., ADLMGYIPLV (Core.sub.131-140) and LLALLSCLTV (Core178-187)), sequences from NS3 (e.g., LLCPAGHAV (NS3.sub.1169-1177) and KLVALGINAV (NS3.sub.1406-1415)), sequences from NS4 (e.g., SLMAFTAAV (NS4.sub.1789-1797) and LLFNILGGWV (NS4.sub.1807-1816)), and sequences from NS5, e.g., ILDSFDPLV (NS5.sub.2252-2260). See U.S. Pat. No. 5,709,995, issued to Chisari et al., col. 3, line 62- col. 4, line 4.

U.S. Pat. No. 6,685,947 to Jackson et al. also discloses helper T-lymphocyte epitopes which are also useful in the practice of the present invention. In addition, helper T-lymphocytes epitopes can be artificial peptides, as disclosed in U.S. Pat. No. 6,713,301, issued to Wang.

Spacer moieties useful in the practice of this invention include any spacer moiety. Such moieties are well known in the art. For instance, useful peptide spacer moieties include gly-gly, as described in U.S. Pat. No. 5,759,551, issued to Ladd et al., col. 9, line 64; the inactive peptides of U.S. Pat. No. 6,613,530, issued to Wienhues et al., col. 3, lines 38-47; and the proline rich flexible hinge spacers disclosed in U.S. Pat. No. 5,683,695, issued to Shen et al. Further, non-peptide spacer moieties are also useful and have the added feature that they are generally protease resistant. Such moieties include, for instance, —O—R—CO—, —NH—R—CO—, —NH—R—NH—, —O—R—NH—, or-, —NH—R—CH₂—, in which R is a saturated or unsaturated hydrocarbon chain optionally substituted and/or interrupted by one or more aromatic radicals or heteroatoms, e.g. a nitrogen atom, an oxygen atom or a sulfur atom, as disclosed in U.S. Pat. Nos. 5,736,146, and 5,869,058, both issued to Cohen et al.

“Synergy” as used herein, results wherever the immunogenic response induced by the immunogen composition is greater than the sum of the responses induced by each separate immunogen within the composition. The synergy in immune response is represented by, but not limited to, the increase in antibody titer elicited by the chimeric immunogen composition over and above the sum of the antibody titers elicited by the component chimeric immunogens when each is administered alone.

“Suppression” as used herein, results whenever the immunogenic response induced by the composition is less than the sum of the responses induced by each separate immunogen within the composition. Suppression of an immune response can be used to treat any disease or condition manifesting an antibody response to a particular antigen. Such diseases or conditions include, for instance, allergic responses, graft rejection, autoimmune diseases, as well as chronic inflammatory diseases.

The particular helper T-lymphocyte epitope combinations that provide the synergistic or suppressive antibody responses desired can be determined by the routine methods taught herein. For example, antibody titers elicited by compositions of the invention that include two or more chimeric immunogens can be compared with the sum of the antibody titers elicited by each component when administered separately.

Similarly, suppressive helper T-lymphocyte epitope combinations can be tested and identified. Where the antibody response that is desired to be suppressed is due to a known antigen, suppressive activity of the chimeric immunogens of the present invention can be tested in combination with the known antigen, by testing each of a variety of chimeric immunogens having different helper T-lymphocyte epitopes, until chimeras that suppress the antibody response to the particular antigen are identified.

Suppression of an antibody response is particularly useful in the treatment of allergies, inflammatory conditions and autoimmune diseases, and in tissue or organ transplantation.

An allergy is a response that occurs when the immune system reacts inappropriately to highly specific agents (allergens) that are otherwise non-pathogenic in the concentrations normally encountered. These agents or allergens contain antigen components that are highly immunogenic in certain individuals among the population that suffer from these specific allergies. Allergens include many common antigens found in natural materials such as tree and grass pollens(e.g. from timothy grass or rye grass), weeds (e.g. ragweed); cockroaches; dust mites; animal dander; latex; honeybee, wasp and fire ant venoms; foods such as peanuts, tree nuts, milk, fish, shellfish, eggs, soy, wheat, honey, cantaloupe, strawberries and tropical fruits. Certain other natural and non-natural antigens may cause allergies in some individuals. These include drugs such as penicillin, anesthetics, serum, some viruses, bacteria, protozoa, and molds; and oils found in poison ivy, poison oak and sumac.

Chronic inflammatory diseases that can be suppressed by the chimeric immunogens of the present invention include systemic lupus erythematosis, chronic rheumatoid arthritis, type 1 diabetes mellitus, type 2 adult onset diabetes, biliary cirrhosis, uveitis, multiple sclerosis and other disorders such as bullous pemphigoid (a chronic, autoimmune, subepidermal, blistering skin disease), sarcoidosis (inflammation of the tissues of the body) and Graves ophthalmopathy (a condition that primarily affects the extraocular muscles. It is closely associated with Graves' disease, an autoimmune disorder that causes hyperthyroidism).

Autoimmune diseases include juvenile-onset or adult-onset diabetes mellitus, multiple sclerosis, and rheumatoid arthritis, liver disease, posterior uveitis, allergic encephalomyelitis, and glomerulonephritis.

Graft rejection can occur following a bone-marrow or an organ transplantation. The host lymphocytes recognize the foreign tissue antigens and begin to produce antibodies directed against one or more antigens of the foreign tissue which leads to rejection of the foreign tissue, known as graft rejection.

The present invention provides a method of eliciting a antibody response to a target antigen in a patient; wherein the method includes: administering to the patient a composition of two or more of the above-described chimeric immunogens having identical immunomimics of the target antigen, wherein each of the immunogen components of the composition includes a different helper T-lymphocyte epitope, and wherein the different helper T-lymphocyte epitopes act synergistically to boost the antibody response to the chimeric immunogens.

The present invention yet further provides a method of suppressing an antibody response to a target antigen in a patient; the method includes: administering to the patient a composition that includes one or more chimeric immunogens as described above, wherein at least one of the immunogens includes a helper T-lymphocyte epitope not found in the target antigen. This method is particularly useful the antibody response to the target antigen is an autoimmune antibody response, an inflammatory antibody response, an allergic antibody response or an antibody response to a tissue or an organ graft.

The present invention also provides a pharmaceutical composition that includes two or more chimeric immunogens for modulating, i.e. synergistically enhancing or suppressing the production of antibodies against a target antigen; wherein the chimeric immunogen includes: i) a helper T-lymphocyte epitope, including an amino acid sequence, fused through ii) a spacer moiety, to iii) an immunomimic of the target antigen, and iv) a pharmaceutically acceptable carrier. The pharmaceutical compositions containing multiple chimeric immunogens of the invention each include a different helper T-lymphocyte epitope. Preferably, the helper T-lymphocyte epitopes of the chimeric immunogens are not found within the target antigen.

Alternatively, the pharmaceutical composition of the invention can include a single chimeric immunogen as described above, for suppressing an existing antibody response, such as an autoimmune antibody response, an inflammatory antibody response, an allergic antibody response or an antibody response to a tissue or an organ graft.

Administration of these chimeric immunogens and compositions containing them, or pharmaceutically acceptable and immunologically effective derivatives thereof, may be via any of the conventionally accepted modes of administration of agents which exhibit immunogenicity. These include subcutaneous, intramuscular, intranasal and oral administration, as well as parenteral or even topical administration.

The compositions used in these pharmaceutical compositions may be in a variety of forms. These include, for example, solid, semi-solid and liquid dosage forms, such as powders, liquid solutions or suspensions, suppositories, injectable and infusible solutions. The preferred form depends on the intended mode of administration and the therapeutic application.

The compositions also will preferably include conventional pharmaceutically acceptable vehicles or carriers and may include other medicinal agents, carriers, adjuvants, excipients, etc., e.g., human serum albumin or plasma preparations. Preferably, the compositions of the invention are in the form of a unit dose. The amount of active compound administered as a vaccination or as a medicament at one time, or over a period of time, will depend on subject being treated, the manner and form of administration, and the judgment of the treating physician. However, an effective dose may be in the range between about 1 microgram (1 ug) and about 10 milligrams (10 mg) of each of the chimeric immunogens of the invention, preferably between about 100 ug and about 2 mg; it being recognized that lower and higher doses may also be useful.

### EXAMPLE I

### GnRH Chimeric Immunogens

The peptide sequences combine a select promiscuous T-helper-epitope through an inserted short spacer peptide (e.g., 4-8 amino acids) with at least one target hormone peptide. Suitable spacers of this invention include but are not limited to the peptides comprising the following amino acid sequence, GPSL (see SEQ ID NO: 5); SSGPSL (SEQ ID NO: 6); and SSGPSLKL (SEQ ID NO: 7), which are inserted in the peptide chimera to isolate the three dimensional folding of the immunogenic peptide from that of the hormone peptide.

Promiscuous Th-epitope moieties from measles virus protein F (MSF) (sequence 288-302 aa, SEQ ID NO: 8), tetanus toxoid (TT) (sequence 947-967 aa, SEQ ID NO: 4, or sequence 830-844 aa, SEQ ID NO: 2) and malaria Plasmodium falciparum CSP protein (sequence 378-398 aa, SEQ ID NO: 3) are used in these constructs. The hormone immunomimic epitopes were attached to the N-terminal or the C-terminus of the spacer as shown below. All mammalian GnRH peptides including the human hormone, have the same sequence. The GnRH hormone immunomimic epitope sequence comprises 1-10 amino acids of mammalian GnRH when attached to the aminoterminal peptide end and comprises 2-10 amino acids of mammalian GnRH when attached to the carboxyterminal peptide end. In addition, an immunomimic peptide comprising 13-16 amino acids of the mammalian GnRH comprise GnRH immunomimics peptides attached to both ends of the spacer, in order to increase the number of available GnRH antigenic epitopes. The different peptide chimera fusion immunogens in terms of antibodies produced are described below.

Peptide 1.

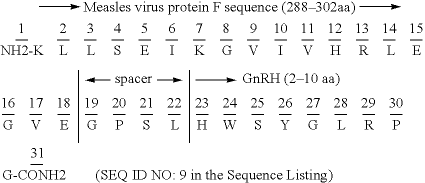

Peptide 2.

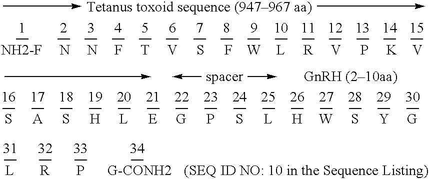

Peptide 3.

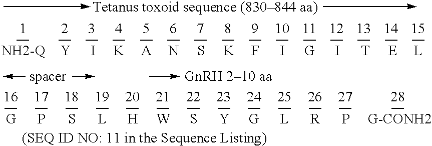

Peptide 4.

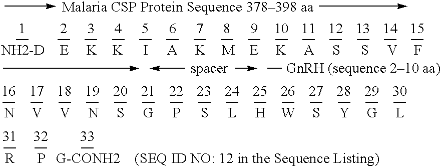

Peptide 5.

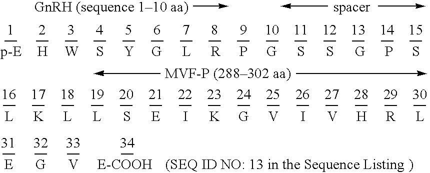

Peptide 6.

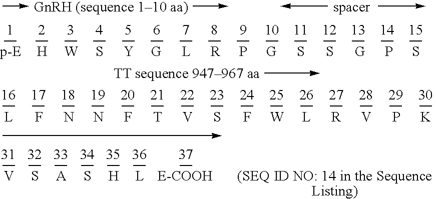

Peptide 7.

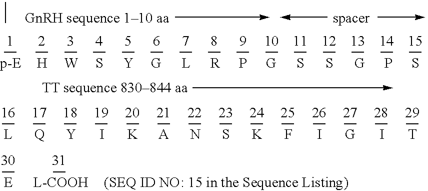

Peptide 8.

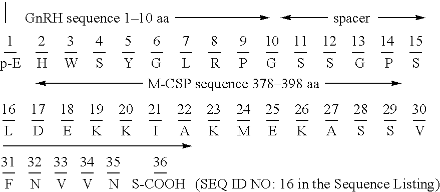

Peptide 9.

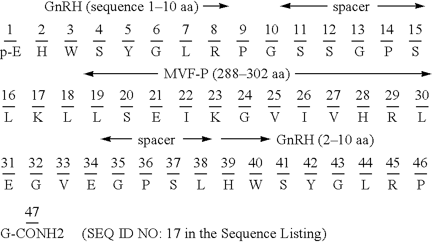

Peptide 10.

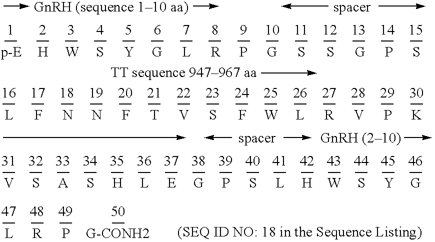

Peptide 11.

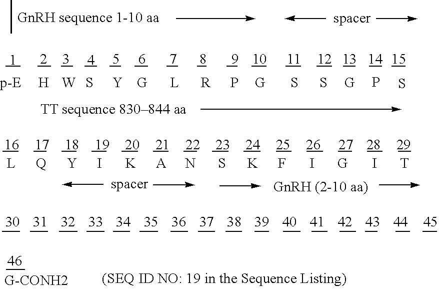

Peptide 12.

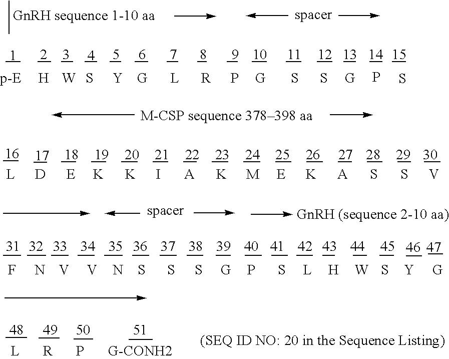

Peptide 13.

(SEQ ID NO: . . . in the Sequence Listing)

GnRH chimeric immunogen 13 (TT-10):
H-Leu-Arg-Val-Asp-Asn-Lys-Asn-Tyr-Phe-Pro-Cys-Arg-
Asp-Gly-Phe-Gly-Ser-Ile-Met-Gln-Gly-Pro-Ser-Leu-
His-Trp-Ser-Tyr-Gly-Leu-Arg-Pro-Gly-NH₂

Peptide 14.

(SEQ ID NO: . . . in the Sequence Listing)

GnRH chimeric immunogen 14 (TT-11):
H-Asn-Glu-Ile-Val-Ser-Tyr-Asn-Thr-Lys-Asn-Lys-Pro-
Leu-Asn-Phe-Asn-Tyr-Ser-Leu-Asp-Gly-Pro-Ser-Leu-
His-Trp-Ser-Tyr-Gly-Leu-Arg-Pro-Gly-NH₂

Peptide 15.

(SEQ ID NO: . . . in the Sequence Listing)

GnRH chimeric immunogen 15 (DT-2):
H-Gln-Ser-Ile-Ala-Leu-Ser-Ser-Leu-Met-Val-Ala-Gln-
Ala-Ile-Pro-Leu-Val-Gly-Glu-Leu-Gly-Pro-Ser-Leu-
His-Trp-Ser-Tyr-Gly-Leu-Arg-Pro-Gly-NH₂

Peptide 16.

(SEQ ID NO: . . . in the Sequence Listing)

GnRH chimeric immunogen 18 (TT-5):
H-Gly-Val-Leu-Leu-Pro-Thr-Ile-Pro-Gly-Lys-Leu-Asp-
Val-Asn-Lys-Ser-Lys-Thr-His-Ile-Gly-Pro-Ser-Leu-
His-Trp-Ser-Tyr-Gly-Leu-Arg-Pro-Gly-NH₂

### EXAMPLE II

### Immunogenicity of Chimeric Immunogens

Immunogenicity tests were performed with five chimeric peptide immunogens against GnRH. Each chimeric peptide contained one region encoding an epitope to be recognized by helper T-cell and a second region encoding an immunomimic of GnRH, to serve as the target for the antibody response. The chimeric peptide immunogens were formulated to deliver 100, 250 or 500 μg doses of peptide with 3 μg norMDP, in a water in oil emulsion. Control immunogens were prepared to deliver 500 μg of mammalian GnRH (1-10) Ser1 peptide (which is normally linked to an immunogenic carrier to impart immunogenicity), with and without norMDP (3 μg), in the same emulsions. The immunogens were given intramuscularly to rabbits in three injections, on days 0, 14 and 42. An ELISA procedure was used to measure the resultant anti-GnRH antibody responses in sera collected at 14-day intervals over the course of the immunization. Injection site reactions were assessed by visual and microscopic evaluations on day 84.

The following materials were used in the immunogenicity tests. The five immunogens of GnRH chimera peptides tested were selected from the aforementioned Peptide 1 through 16.
1. GnRH chimera 1 {MVF (Measles Virus Protein F)} “Peptide 1” (MW 3427.17)
2. GnRH chimera 2 {TT-3 (Tetanus Toxoid Epitope 3)} “Peptide 2” (MW 3886.52)
3. GnRH chimera 3 {TT-2 (Tetanus Toxoid Epitope 2)} “Peptide 3” (MW 3132.6)
4. GnRH chimera 4 {MCSP (Malaria Circumsporozoite Protein)} “Peptide 4” (MW 3632.2)
5. GnRH chimera 6 (TT-3, N-ter GnRH) “Peptide 6” (MW 4172.7)
6. D17 Peptide (“GnRH (1-10) Ser 1”)

For testing the GnRH chimeric peptide immunogens were formulated at concentrations listed below in Table 1. Each injection volume was 0.2 ml/dose (see Table 2).

TABLE 1
GnRH Chimera and Control Immunogen Formulations

| | | Concentration | | Concentration | |
|---|---|---|---|---|---|
| | | of Peptide in | Peptide | of norMDP in | norMDP |
| | Chimeric | Emulsion | Dose | Emulsion | Dose |
| Immunogen | Peptide | (mg/ml) | (μg/dose) | (mg/ml) | (μg/dose) |
| A | Peptide 1 | 2.5 | 500 | 0.015 | 3 |
| B | Peptide 2 | 2.5 | 500 | 0.015 | 3 |
| C | Peptide 2 | 1.25 | 250 | 0.015 | 3 |
| D | Peptide 2 | 0.5 | 100 | 0.015 | 3 |
| E | Peptide 3 | 1.25 | 500 | 0.0072 | 3 |
| F | Peptide 4 | 2.5 | 500 | 0.015 | 3 |
| G | Peptide 6 | 2.5 | 500 | 0.015 | 3 |
| H | Peptide 2 | 2.5 | 500 | 0.015 | 3 |
| I | Peptide 3 | 1.25 | 500 | 0.0072 | 3 |
| J | Peptide 2 & 3 | 0.625, each | 250, each | 0.0072 | 3 |
| | | peptide | peptide | | |
| K | D17 Peptide | 2.5 | 500 | 0.015 | 3 |
| L | D17 peptide | 2.5 | 500 | — | — |

The GnRH chimeric immunogenic compositions and control immunogens were formulated under clean conditions in the combinations shown in Table 1. The test materials were sterile bottled and stored under refrigeration (2-8° C.).

New Zealand White female rabbits were immunized with GnRH chimera and control immunogens as shown in Table 2. Injections were given to each rabbit on days 0, 14 and 42 in dose volumes of either 0.2 ml or 0.4 ml. All immunogens were given IM, at injection sites tattooed for later identification.

To assess immunogenicity, sera were obtained from each rabbit every 14 days until day 84. Anti-GnRH antibody titers were measured in the sera samples by a direct binding ELISA. All values, with the exception of those for immunogen 6, are expressed relative to a reference standard rabbit anti-GnRH serum reference titer of 5,000. Titers of sera against Immunogen 6 (Peptide 6 N-terminal specific antibodies) were expressed relative to the reference standard rabbit anti-GnRH serum Ser 10(11) reference titer of 20,000.

Although the original study had two rabbit groups, the protocol was later amended to add two more groups (n=4), 3 and 4, with amounts of 250 fig and 100 μg of GnRH chimera 2 (TT-3) (Peptide 2), each with 3 μg of norMDP.

TABLE 2
Immunization Schedule

| | | | Injection |
|---|---|---|---|
| Rabbit | | | Volume |
| Group Number | N* | Peptide(s) | (ml/dose) |
| 1 | 4 | Peptide 1 500 μg | 0.2 |
| 2 | 4 | Peptide 2 500 μg | 0.2 |
| 3 | 4 | Peptide 2 250 μg | 0.2 |
| 4 | 4 | Peptide 2 100 μg | 0.2 |
| 5 | 4 | Peptide 3 500 μg | (2 × 0.2/site)** |
| 6 | 4 | Peptide 4 500 μg | 0.2 |
| 7 | 4 | Peptide 6 500 μg | 0.2 |
| 8 | 10 | Peptide 2 500 μg | 0.2 |
| 9 | 10 | Peptide 3 500 μg | (2 × 0.2/site)** |
| 10 | 6 | Peptides 2 & 3, 250 μg each | (2 × 0.2/site)** |
| 11 | 4 | D17 peptide (500 μg) with | 0.2 |
| | | norMDP | |
| 12 | 4 | D17 peptide 500 μg | 0.2 |

*N = number of rabbits per group
**Peptide 3 did not dissolve at higher concentrations, therefore injection volumes were doubled to deliver 500 μg/dose of total peptide.

Since GnRH chimera peptide 3 (“Peptide 3”) (TT-2) was not found soluble at 9.412 mg/ml in aqueous phase, the original protocol was amended to reduce the concentration in half (4.706 mg/ml) and double the dose volume to maintain 0.2 ml volume per injection (2×0.2 ml/site). Injection #3 was delivered on day 42.

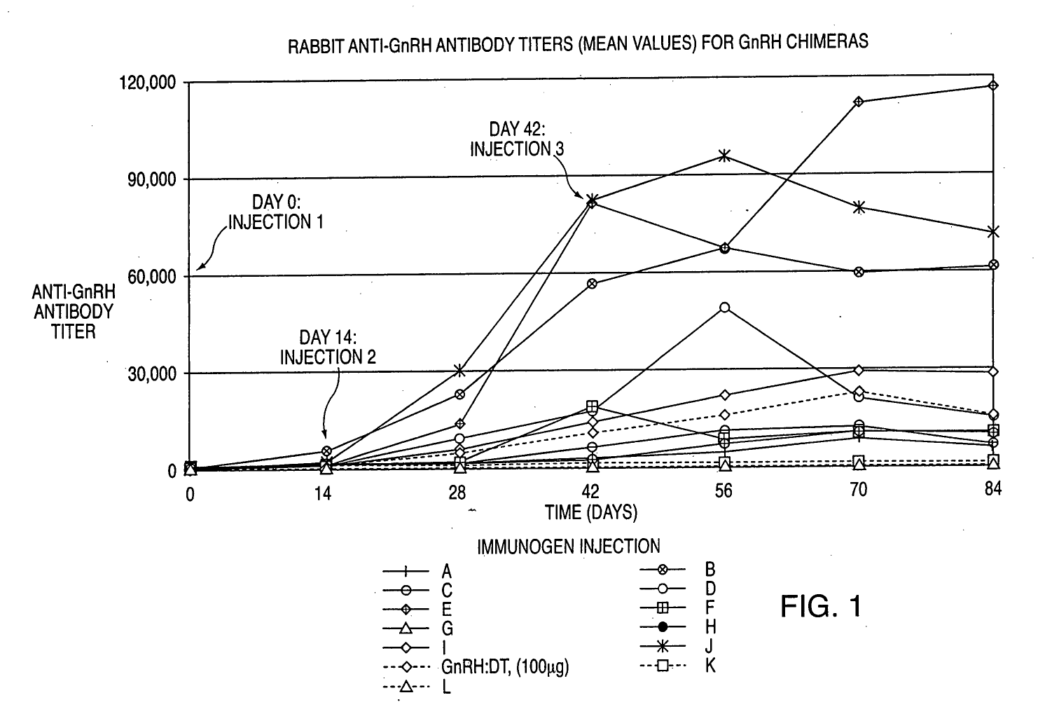

Titers obtained for the individual serum samples are given in Table 3A/B/C, and mean titers for all groups are plotted in FIG. 1, respectively. In the initial tests, all rabbits responded to the chimera peptides with the production of anti-GnRH antibody titers. Peptide 3 or GnRH chimera 3 (TT-2) induced significantly higher antibody titers in comparison with the other chimera peptides. Peptide 2 or Chimera 2 was most immunogenic at the 500 μg dose (Immunogen B), with the 100 μg (Immunogen D) and 250 μg (Immunogen C) doses inducing weaker titers. Chimeras 2 (Immunogen B) and 3 (Immunogen E) induced high antibody titers in the initial tests (n=4) relative to titers induced by GnRH:DT; however, these titers were lower in the repeat studies (n=10, Immunogen H where the response rate was quite variable, and Immunogen I, respectively).

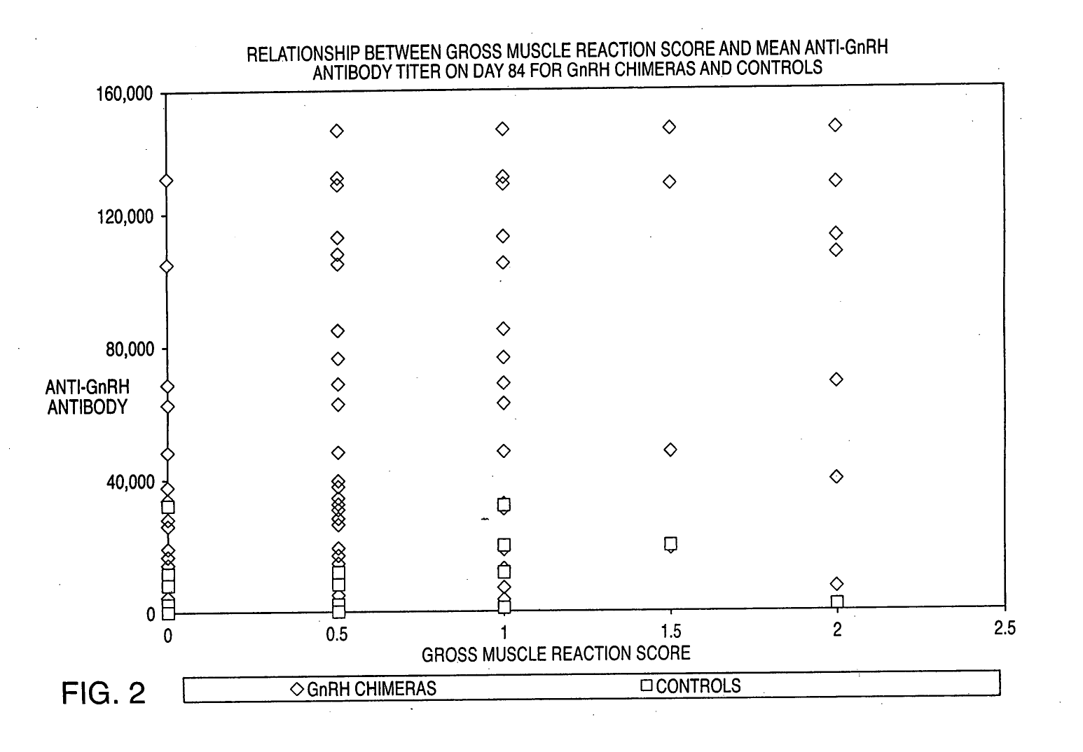

A combination of Chimeras 2 and 3 (Immunogen J), at 250 fig dose of each (half the dose used in rabbits injected with the individual peptides) induced high titers of anti-GnRH antibody. Chimeras 1 (Immunogen A), 4 (Immunogen F) and 6 (Immunogen G) were not as potent as the GnRH:DT conjugate formulated in Montanide ISA 703 (as historical control included in FIGS. 1 and 2). It should be noted that Peptide 6 or GnRH chimera 6 (TT-3 in aminoterminal position) titers were measured using an N-terminus specific reference standard, therefore a statistical comparison of these titers with other chimera peptides was not performed. Nevertheless, Peptide 6 was concluded not to be an effective immunogen. Very low anti-GnRH antibody titers were induced by DI 7 peptide adjuvanted with norMDP (Immunogen K), while without norMDP (Immunogen L), the D17 peptide emulsion was not immunogenic.

Gross pathology of injection sites was assessed on all rabbits on day 84. The evaluation was scored on a scale of 0-3, where a score of 0 indicated normal tissue appearance and 3 indicated the presence of extensive tissue inflammation. Scores of 1 or 2 were judged intermediate levels of local reaction.

TABLE 3A
Anti-GnRH Antibody Titers for GnRH Chimeras

| | Injection | Injection 1 | Injection 2 | | Injection 3 | | | |
|---|---|---|---|---|---|---|---|---|
| | → | (Day 0) | (Day 14) | | (Day 42) | | | |
| | Rabbit | Day | Day | Day | Day | Day | Day | Day |
| Immunogen | # | 0 | 14 | 28 | 42 | 56 | 70 | 84 |
| A | 1 | 0 | 274 | 3,276 | 8,845 | 12,500 | 20,600 | 13,200 |
| | 2 | 0 | 0 | 636 | 2,193 | 4,667 | 13,400 | 8,249 |
| | 3 | 0 | 0 | 198 | 512 | 731 | 1,392 | 1,166 |
| | 4 | 0 | 0 | 0 | 0 | 0 | 0 | 0 |
| | Mean | 0 | 69 | 1,028 | 2,888 | 4,475 | 8,848 | 5,654 |
| | Median | 0 | 0 | 417 | 1,353 | 2,699 | 7,396 | 4,708 |
| | S.D. | 0 | 137 | 1,522 | 4,081 | 5,729 | 9,878 | 6,213 |
| B | 5 | 0 | 8,201 | 20,500 | 37,400 | 34,500 | 62,100 | 76,800 |
| | 6 | 0 | 12,400 | 46,400 | 81,200 | 134,000 | 93,100 | 108,000 |
| | 7 | 0 | 507 | 22,300 | 91,800 | 75,000 | 50,600 | 28,400 |
| | 8 | 0 | 589 | 2,085 | 16,100 | 24,800 | 31,800 | 32,700 |
| | Mean | 0 | 5,424 | 22,821 | 56,625 | 67,075 | 59,400 | 61,475 |
| | Median | 0 | 4,395 | 21,400 | 59,300 | 54,750 | 56,350 | 54,750 |
| | S.D. | 0 | 5,886 | 18,181 | 35,838 | 49,632 | 25,705 | 37,953 |
| C | 9 | 0 | 0 | 536 | 1,325 | 6,631 | 7,267 | 5,033 |
| | 10 | 0 | 0 | 1,240 | 3,551 | 19,700 | 19,600 | 7,886 |
| | 11 | 0 | 0 | 719 | 16,800 | 12,800 | 16,800 | 11,200 |
| | 12 | 0 | 0 | 454 | 2,671 | 5,017 | 5,844 | 3,692 |
| | Mean | 0 | 0 | 737 | 6,087 | 11,037 | 12,378 | 6,953 |
| | Median | 0 | 0 | 628 | 3,111 | 9,716 | 12,034 | 6,460 |
| | S.D. | 0 | 0 | 353 | 7,201 | 6,679 | 6,844 | 3,328 |
| D | 13 | 0 | 2,952 | 8,320 | 869 | 87,200 | 47,300 | 39,700 |
| | 14 | 0 | 841 | 21,600 | 57,500 | 93,000 | 25,100 | 11,800 |
| | 15 | 0 | 141 | 1,759 | 4,373 | 7,732 | 6,670 | 5,198 |
| | 16 | 0 | 0 | 5,220 | 7,044 | 7,363 | 6,120 | 4,731 |
| | Mean | 0 | 984 | 9,225 | 17,447 | 48,824 | 21,298 | 15357 |
| | Median | 0 | 491 | 6,770 | 5,709 | 47,466 | 15,885 | 8,499 |
| | S.D. | 0 | 1,363 | 8,674 | 26,822 | 47,721 | 19,450 | 16,546 |
| E | 17 | 0 | 1,382 | 15,500 | 140,000 | 79,900 | 136,000 | 105,000 |
| | 18 | 0 | 264 | 13,200 | 50,800 | 41,700 | 120,000 | 145,000 |
| | 19 | 0 | 471 | 13,000 | 98,900 | 95,700 | 111,000 | 131,000 |
| | 20 | 0 | 2,317 | 13,400 | 35,900 | 52,800 | 80,500 | 85,100 |
| | Mean | 0 | 1,109 | 13,775 | 81,400 | 67,525 | 111,875 | 116,525 |
| | Median | 0 | 927 | 13,300 | 74,850 | 66,350 | 115,500 | 118,000 |
| | S.D. | 0 | 941 | 1,162 | 47,423 | 24,703 | 23,332 | 26,713 |
| F | 21 | 0 | 296 | 3,189 | 2,638 | 2,165 | 2,751 | 3,365 |
| | 22 | 0 | 0 | 441 | 5,920 | 4,912 | 8,760 | 12,200 |
| | 23 | 0 | 0 | 484 | 6,350 | 6,333 | 7,900 | 7,512 |
| | 24 | 0 | 0 | 3,556 | 60,300 | 20,400 | 24,300 | 18,700 |
| | Mean | 0 | 74 | 1,918 | 18,802 | 8,453 | 10,928 | 10,444 |
| | Median | 0 | 0 | 1,837 | 6,135 | 5,623 | 8,330 | 9,856 |
| | S.D. | 0 | 148 | 1,687 | 27,716 | 8,151 | 9,301 | 6,582 |

TABLE 3B
Anti-GnRH Antibody Titers for GnRH Chimeras (continued)

| | Injection | Injection 1 | Injection 2 | | Injection 3 | | | |
|---|---|---|---|---|---|---|---|---|
| | → | (Day 0) | (Day 14) | | (Day 42) | | | |
| | Rabbit | Day | Day | Day | Day | Day | Day | Day |
| Immunogen | # | 0 | 14 | 28 | 42 | 56 | 70 | 84 |
| G | 25 | 0 | 0 | 0 | 0 | 105 | 640 | 1,165 |
| | 26 | 0 | 0 | 0 | 0 | 0 | 131 | 141 |
| | 27 | 0 | 0 | 0 | 166 | 914 | 3,554 | 3,830 |
| | 28 | 0 | 0 | 0 | 191 | 387 | 1,265 | 1,510 |
| | Mean | 0 | 0 | 0 | 89 | 352 | 1,398 | 1,662 |
| | Median | 0 | 0 | 0 | 83 | 246 | 953 | 1,338 |
| | S.D. | 0 | 0 | 0 | 104 | 409 | 1,511 | 1,558 |
| H | 29 | 0 | 0 | 0 | 0 | 208 | 708 | 693 |
| | 30 | 0 | 0 | 1,257 | 1,475 | 2,800 | 2,374 | 2,313 |
| | 31 | 0 | 0 | 0 | 0 | 0 | 0 | 0 |
| | 32 | 0 | 0 | 0 | 147 | 1,319 | 2,051 | 1,559 |
| | 33 | 0 | 204 | 3,713 | 8,696 | 11,900 | 14,100 | 11,200 |
| | 34 | 0 | 0 | 413 | 480 | ** | 16,900 | 14,700 |
| | 35 | 0 | 0 | 366 | 326 | 1,879 | 3,462 | 3,022 |
| | 36 | 0 | 0 | 0 | 0 | 200 | 410 | 555 |
| | 37 | 0 | 0 | 163 | 774 | 2,825 | 4,677 | 5,109 |
| | 38 | 0 | 2,787 | 8,027 | 7,742 | 41,700 | 63,200 | 62,900 |
| | Mean | 0 | 299 | 1,394 | 1,964 | 6,981 | 10,788 | 10,205 |
| | Median | 0 | 0 | 265 | 403 | 1,879 | 2,918 | 2,668 |
| | S.D. | 0 | 877 | 2,597 | 3,335 | 13,523 | 19,319 | 19,149 |
| I | 39 | 0 | 0 | 228 | 877 | 7,841 | 12,200 | 9,998 |
| | 40 | 0 | 0 | 2,568 | 5,522 | 27,000 | 29,600 | 17,000 |
| | 41 | 0 | 895 | 7,474 | 31,400 | 29,500 | 46,300 | 34,500 |
| | 42 | 0 | 0 | 1,560 | 3,280 | 10,800 | 12,000 | 11,500 |
| | 43 | 0 | 222 | 3,510 | 16,600 | 20,600 | 31,300 | 26,500 |
| | 44 | 0 | 0 | 5,825 | 22,500 | 27,000 | 36,200 | 37,900 |
| | 45 | 0 | 1,249 | 24,300 | 39,300 | 65,000 | 67,700 | 69,100 |
| | 46 | 0 | 498 | 5,208 | 7,243 | 8,877 | 13,500 | 16,800 |
| | 47 | 0 | 0 | 2,091 | 5,509 | 10,100 | 19,200 | 18,300 |
| | 48 | 0 | 0 | 4,072 | 7,937 | 14,600 | 26,300 | 48,400 |
| | Mean | 0 | 286 | 5,684 | 14,017 | 22,132 | 29,430 | 29,000 |
| | Median | 0 | 0 | 3,791 | 7,590 | 17,600 | 27,950 | 22,400 |
| | S.D. | 0 | 452 | 6,886 | 13,061 | 17,164 | 17,535 | 18,782 |
| J | 49 | 0 | 219 | 4,179 | 33,900 | 81,500 | 85,300 | 113,000 |
| | 50 | 0 | 8,659 | 100,000 | 193,000 | 242,000 | 169,000 | 129,000 |
| | 51 | 0 | 305 | 14,800 | 89,500 | 91,300 | 97,800 | 69,000 |
| | 52 | 0 | 1,071 | 11,000 | 26,600 | 30,600 | 27,500 | 19,300 |
| | 53 | 0 | 554 | 16,300 | 64,000 | 32,500 | 31,400 | 31,100 |
| | 54 | 0 | 1,940 | 32,700 | 86,400 | 70,500 | 65,600 | 68,800 |
| | Mean | 0 | 2,125 | 29,830 | 82,333 | 91,400 | 79,433 | 71,700 |
| | Median | 0 | 813 | 15,550 | 75,200 | 76,000 | 75,450 | 68,900 |
| | S.D. | 0 | 3,263 | 35,647 | 60,172 | 77,950 | 52,134 | 43,356 |
| K | C1 | 0 | 746 | 1,515 | 2,201 | 1,918 | 2,074 | 1,913 |
| | C2 | 0 | 0 | 0 | 0 | 0 | 0 | 0 |
| | C3 | 0 | 134 | 590 | 953 | 998 | 1,238 | 1,768 |
| | C4 | 0 | 323 | 2,279 | 1,345 | 1,225 | 1,640 | 987 |
| | Mean | 0 | 301 | 1,096 | 1,125 | 1,035 | 1,238 | 1,167 |
| | Median | 0 | 229 | 1,053 | 1,149 | 1,112 | 1,439 | 1,378 |
| | S.D. | 0 | 325 | 1,005 | 913 | 793 | 893 | 878 |
| L | C5 | 0 | 0 | 0 | 0 | 0 | 0 | 0 |
| | C6 | 0 | 0 | 0 | 0 | 0 | 0 | 0 |
| | C7 | 0 | 0 | 0 | 0 | 107 | 0 | 0 |
| | C8 | 0 | 0 | 0 | 0 | 0 | 0 | 0 |
| | Mean | 0 | 0 | 0 | 0 | 27 | 0 | 0 |
| | Median | 0 | 0 | 0 | 0 | 0 | 0 | 0 |
| | S.D. | 0 | 0 | 0 | 0 | 54 | 0 | 0 |

TABLE 3C
Anti-GnRH Antibody Titers for GnRH Chimeras

| | Injection | Injection 1 | Injection 2 | | Injection 3 | | | |
|---|---|---|---|---|---|---|---|---|
| | → | (Day 0) | (Day 14) | | (Day 42) | | | |
| | Rabbit | Day | Day | Day | Day | Day | Day | Day |
| Immunogen | # | 0 | 14 | 28 | 42 | 56 | 70 | 84 |
| Control | C9 | 0 | 475 | 7,210 | 11,400 | 8,812 | 8,762 | 8,338 |
| | C10 | 0 | 1,588 | 9,253 | 20,100 | 28,500 | 34,800 | 32,200 |
| GnRHDT | C11 | 0 | 0 | 4,593 | 17,700 | 25,100 | 35,400 | 19,800 |
| Conjugate in | C12 | 0 | 194 | 3,647 | 7,900 | 13,900 | 12,900 | 11,800 |
| Emulsion = | C13 | 0 | 169 | 1,565 | 2,559 | 4,752 | 7,204 | 7,115 |
| 0.5 mg/ml | C14 | 0 | 651 | 3,965 | 3,755 | 8,277 | 13,700 | 7,179 |
| Conjugate | C15 | 0 | 123 | 2,785 | 2,627 | 4,198 | 5,218 | 3,891 |
| Dose = 100 μg | C16 | 0 | 353 | 4,910 | 13,800 | 26,700 | 43,600 | 30,600 |
| Dose Volume = | C17 | 0 | 333 | 8,573 | 25,100 | 30,300 | 57,400 | 26,200 |
| 0.2 ml | C18 | 0 | 188 | 2,171 | 2,622 | 7,314 | 8,207 | 8,404 |
| | Mean, | 0 | 407 | 4,867 | 10,756 | 15,785 | 22,719 | 15,553 |
| | Group 5 | | | | | | | |
| | Median, | 0 | 264 | 4,279 | 9,650 | 11,356 | 13,300 | 10,102 |
| | Group 5 | | | | | | | |
| | S.D. | 0 | 455 | 2,653 | 8,216 | 10,617 | 18,486 | 10,695 |

*test titers are read at 20,000 titer of a reference standard lot

The score data are summarized in Table 4, indicating that most of the visual injection site scores ranged from 0 to 1, indicating that the immunogens were generally well tolerated. Histologic readings of the injection site biopsies which were performed as of day 84 were in accord with the gross evaluation.

These experiments demonstrated that chimera peptides carrying a T-lymphocyte epitope and expressing an immunomimic of GnRH can be used to induce potent anti-GnRH antibody responses. Peptides bearing TT-2 and TT-3 T-lymphocyte epitopes, derived from TT, were more effective than the T-lymphocyte epitopes derived from MVF and MCSP. A combination of the TT-2 and TT-3 bearing chimeras was particularly effective. Most injection site reactions were of an acceptable level. Overall, the response compared favorably with those induced by the GnRH:DT (previously named, D17-DT) conjugate, indicating that the synthetic peptides could potentially enhance the choice of effective immunogens and perhaps even replace the conjugate method for producing an active component of the GnRH immunogen.

TABLE 4
Reaction Scores

| | MEAN REACTION SCORES | | | | | | REACTION SCORES > 1 | | | | | |
|---|---|---|---|---|---|---|---|---|---|---|---|---|
| | Injection 1 | | Injection 2 | | Injection 3 | | Injection 1 | | Injection 2 | | Injection 3 | |
| Immunogen | Site 1 | Site 2 | Site 1 | Site 2 | Site 1 | Site 2 | Site 1 | Site 2 | Site 1 | Site 2 | Site 1 | Site 2 |
| A | 0 | | 0.5 | | 0.5 | | 0 | | 0 | | 0 | |
| B | 0.4 | | 1.1 | | 1.1 | | 0 | | 1 | | 1 | |
| C | 0.1 | | 0.5 | | 0.5 | | 0 | | 0 | | 0 | |
| D | 0.3 | | 0.4 | | 1.0 | | 0 | | 0 | | 1 | |
| E | 0.6 | 0.3 | 0.9 | 0.6 | 0.8 | 1.3 | 0 | 0 | 1 | 0 | 0 | 1 |
| F | 0.5 | | 1.1 | | 1.1 | | 0 | | 1 | | 1 | |
| G | 0.1 | | 0.3 | | 0.8 | | 0 | | 0 | | 0 | |
| H | 0.1 | | 0.3 | | 0.4 | | 0 | | 0 | | 0 | |
| I | 0 | 0.4 | 0.1 | 0.5 | 0.6 | 0.7 | 0 | 0 | 0 | 0 | 1 | 1 |
| J | 0 | 0.4 | 0.5 | 0.5 | 1.0 | 1.3 | 0 | 0 | 0 | 0 | 1 | 2 |
| K | 0.4 | | 0.4 | | 1.0 | | 0 | | 0 | | 1 | |
| L | 0.3 | | 0 | | 0.3 | | 0 | | 0 | | 0 | |
| Conjugate | 0.4 | | 0.6 | | 0.9 | | 0 | | 0 | | 1 | |
| Ctl. | | | | | | | | | | | | |

### EXAMPLE III

### Modulated Antibody Response By Mixtures of Chimeric Immunogens

The chimeric peptide immunogens were formulated in Montanide ISA 703 with 3 μg of norMDP singly (500 μg dose) or as mixtures of three chimeric immunogens, including chimeric immunogens 2 and 3 (600 μg dose total peptide). One formulation of all six peptides in a single mixture was also prepared (600 μg total dose). See Table 5.

The six GnRH chimeric immunogens tested were selected from the GnRH chimeric peptides 1-16. The materials used in the immunogenicity tests are shown below, including the sequences of the GnRH Chimeric Immunogens (the TT epitope in parenthesis and the GnRH epitope underlined.)
1. GnRH chimeric immunogen 2 {TT-3 (Tetanus Toxoid Epitope 3)} (MW 3886.5)
2. GnRH chimeric immunogen 3 {TT-2 (Tetanus Toxoid Epitope 2)} (MW 3132.6)
3. GnRH chimeric immunogen 13 (Tetanus Toxoid Epitope 10) (MW 3771.1)
4. GnRH chimeric immunogen 14 (Tetanus Toxoid Epitope 11) (MW 3781.9)
5. GnRH chimeric immunogen 15 (Diphtheria Toxoid Epitope 2) (MW 3448)
6. GnRH chimeric immunogen 18 (Tetanus Toxoid Epitope 5) (MW 3538.6)
7. NorMuramyl dipeptide (NorMDP) (Peninsula Laboratories, Inc., USA).
8. Montanide ISA 703 (Seppic, France).
9. Water for Injection (WFI) (Butler, USA).

The GnRH chimeric immunogens were formulated under clean conditions in the combinations shown in Table 5. Using Montanide ISA 703, 70:30 oil:aqueous phase (wt:wt), water-in-oil emulsions were prepared using the Hand Mixing method. WFI was used as the diluent to prepare aqueous phases for GnRH chimeric immunogens. The test materials were bottled in sterile multi-dose crimp cap vials and stored in the laboratory under refrigeration (2-8° C.) until they were delivered to the animal facility where they were stored under refrigeration (2-10° C.) until used.

New Zealand White Female rabbits (Animal Pharm Services, Inc., San Francisco, Calif.) were immunized with GnRH chimeric immunogens as shown in Table 6. Injections were given to each rabbit on days 0, 14 and 42 in dose volumes of either 0.2 ml or 0.4 ml. The chimeric immunogens were administered intramuscularly to rabbits in three injections on days 0, 14 and 42. Each injection site was tattooed for later identification. An ELISA was used to measure the resultant anti-GnRH antibody responses in sera collected at 14-day intervals over the course of the studies. All rabbits were assessed for injection site reactions on day 84, and biopsy specimens were collected from 2 rabbits/group for histopathology analyses.

TABLE 5
GnRH Chimeric Immunogen Formulations

| | | Concentration of | Dose of | Concentration of | |
|---|---|---|---|---|---|
| Immunogen | Chimeric | Peptide(s) in | Peptide(s) | norMDP in | Dose of |
| Lot Number | Immunogen(s) | Emulsion (mg/ml) | (μg/dose) | Emulsion (mg/ml) | norMDP(μg/dose) |
| A | GnRH chimeric | 1.25 total (0.625 each) | 500 total (250 each) | 0.0075 | 3 |
| | immunogen 2 (TT-3) | | | | |
| | and 3 (TT-2) | | | | |
| 3-A | GnRH chimeric | 2.5 | 500 | 0.015 | 3 |
| | immunogen 13 (TT-10) | | | | |
| 3-B | GnRH chimeric | 2.5 | 500 | 0.015 | 3 |
| | immunogen 14 (TT-11) | | | | |
| 3-C | GnRH chimeric | 1.25 | 500 | 0.0075 | 3 |
| | immunogen 15 (DT-2) | | | | |
| 3-D | GnRH chimeric | 2.5 | 500 | 0.015 | 3 |
| | immunogen 18 (TT-5) | | | | |
| 3-E | GnRH chimeric | 3 total (1.5 each) | 600 total (300 each) | 0.015 | 3 |
| | immunogens 2 (TT- | | | | |
| | 3) and 3 (TT-2) | | | | |
| 3-F | GnRH chimeric | 3 total (1 each) | 600 total (200 each) | 0.015 | 3 |
| | immunogens 2 (TT- | | | | |
| | 3), 3 (TT-2) and 13 | | | | |
| | (TT-10) | | | | |
| 3-G | GnRH chimeric | 3 total (1 each) | 600 total (200 each) | 0.015 | 3 |
| | immunogens 2 (TT- | | | | |
| | 3), 3 (TT-2) and 14 | | | | |
| | (TT-11) | | | | |
| 3-H | GnRH chimeric | 1.5 total (0.5 each) | 600 total (200 each) | 0.0075 | 3 |
| | immunogens 2 (TT- | | | | |
| | 3), 3 (TT-2) and 15 | | | | |
| | (DT-2) | | | | |
| 3-I | GnRH chimeric | 3 total (1 each) | 600 total (200 each) | 0.015 | 3 |
| | immunogens 2 (TT- | | | | |
| | 3), 3 (TT-2) and 18 | | | | |
| | (TT-5) | | | | |
| 3-J | GnRH chimeric | 1.5 total (0.25 each) | 600 total (100 each) | 0.0075 | 3 |
| | immunogens 2 (TT- | | | | |
| | 3), 3 (TT-2) and 13 | | | | |
| | (TT-10), 14 (TT-11), | | | | |
| | 15 (DT-2) & 18 (TT- | | | | |
| | 5) | | | | |
| 4-A | GnRH chimeric | 1.5 total (0.75 each) | 600 total (300 each) | 0.0075 | 3 |
| | immunogens 2 (TT- | | | | |
| | 3) and 3 (TT-2) | | | | |
| 4-B | GnRH chimeric | 3 total (1.5 each) | 600 total (300 each) | 0.015 | 3 |
| | immunogens 2 (TT- | | | | |
| | 3) and 3 (TT-2) | | | | |
| 4-C | GnRH chimeric | 3 total (1.5 each) | 600 total (300 each) | 0.015 | 3 |
| | immunogens 2 (TT- | | | | |
| | 3) and 3 (TT-2) | | | | |

TABLE 6
Immunization Agenda for GnRH Chimeric Immunogens

| | | | | Injection |
|---|---|---|---|---|
| Study and/or Group | | | Immunogen | Volume |
| Number | N* | Chimeric Immunogen(s) | Lot Number | (ml/dose) |
| Study 2 | 10 | Chimeric immunogen 2 (TT-3) & | 2 | (2 × 0.2/site)** |
| | | 3 (TT-2) | | |
| Study 3; Group 1 | 5 | Chimeric immunogen 13 (TT-10) | 3-A | 0.2 |
| Study 3; Group 2 | 5 | Chimeric immunogen 14 (TT-11) | 3-B | 0.2 |
| Study 3; Group 3 | 5 | Chimeric immunogen 15 (DT-2) | 3-C | (2 × 0.2/site)** |
| Study 3; Group 4 | 5 | Chimeric immunogen 18 (TT-5) | 3-D | 0.2 |
| Study 3; Group 5 | 5 | Chimeric immunogen 2 (TT-3) & | 3-E | 0.2 |
| | | 3 (TT-2) | | |
| Study 3; Group 6 | 5 | Chimeric immunogen 2 (TT-3), 3 | 3-F | 0.2 |
| | | (TT-2) & 13 (TT-10) | | |
| Study 3; Group 7 | 5 | Chimeric immunogen 2 (TT-3), 3 | 3-G | 0.2 |
| | | (TT-2) & 14 (TT-11) | | |
| Study 3; Group 8 | 5 | Chimeric immunogen 2 (TT-3), 3 | 3-H | (2 × 0.2/site)** |
| | | (TT-2) & 15 (DT-2) | | |
| Study 3; Group 9 | 5 | Chimeric immunogen 2 (TT-3), 3 | 3-I | 0.2 |
| | | (TT-2) & 18 (TT-5) | | |
| Study 3; Group 10 | 5 | Chimeric immunogen 2 (TT-3), 3 | 3-J | (2 × 0.2/site)** |
| | | (TT-2), 13 (TT-10), 14 (TT-11), 15 | | |
| | | (DT-2) & 18 (TT-5) | | |
| Study 3; Group 1 | 5 | Chimeric immunogen 2 (TT-3) & | 4-A | (2 × 0.2/site)** |
| | | 3 (TT-2) | | |
| Study 3; Group 2 | 5 | Chimeric immunogen 2 (TT-3) & | 4-B | 0.2 |
| | | 3 (TT-2) | | |
| Study 3; Group 3 | 5 | Chimeric immunogen 2 (TT-3) & | 4-C | 0.2 |
| | | 3 (TT-2) | | |

*N = number of rabbits per group
**Some Chimeric immunogens did not dissolve at desired concentrations, therefore injection volumes were doubled to maintain the same doses of total peptide.

To assess immunogenicity, sera were obtained from each rabbit every 14 days until day 84, when the rabbits were euthanized. Anti-GnRH antibody titers were measured in the sera samples by a direct binding ELISA. All values are expressed relative to a reference standard rabbit anti-GnRH C-terminal-specific serum, a standard lot with a reference titer of 5,000.

Gross injection site pathology was assessed in all rabbits on day 84. The injection sites were located by the tattoos. All tissues were evaluated for gross pathology on a scale of 0-3, where a score of 0 indicated that the tissues appeared normal, and a score of 3 indicated the presence of an extensive inflammatory reaction throughout the tissues. Scores of 1 and 2 represent intermediate levels of local reaction. After grading the injection sites for gross pathology, injection site biopsies were taken for histopathology from two animals per group. Biopsy specimens were provided to a pathologist for assessment of histopathology.
Study 3 was amended as follows:
GnRH chimeric peptide 15 was not soluble at 9.412 mg/ml in aqueous phase. Therefore the original study protocol was amended to reduce the concentration to half (4.706 mg/ml) and double the dose volumes (2×0.2 ml/site) for Groups 3, 8 and 10.

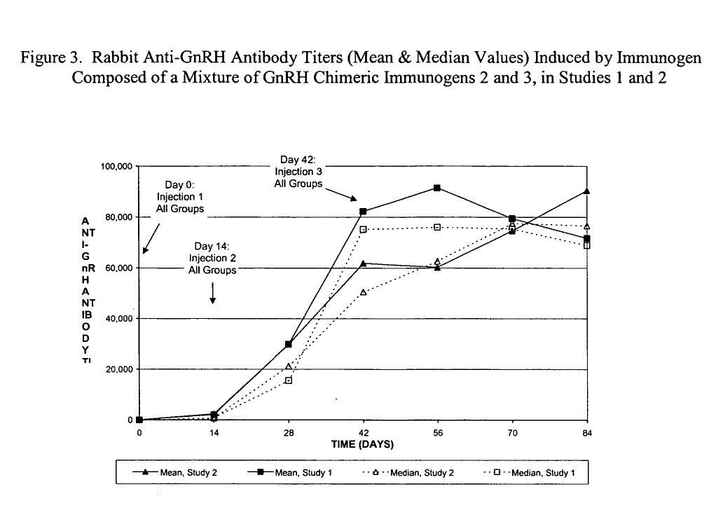

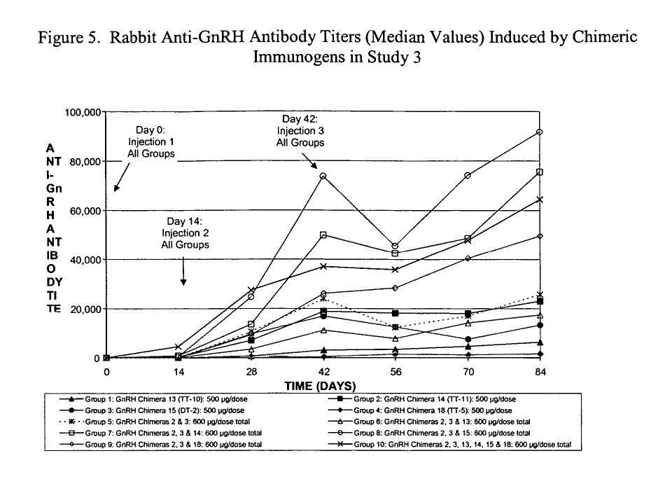

Titers obtained for the individual serum samples are given in Table 7 A/B/C. Mean and median titers for all studies/groups are plotted in FIGS. 3 through 5.

TABLE 7A
Anti-GnRH Antibody Titers for GnRH Chimeric Immunogens: Studies 1 and 2

| | Injection | Injection 1 | Injection 2 | | Injection 3 | | | |
|---|---|---|---|---|---|---|---|---|
| | --> | (Day 0) | (Day 14) | | (Day 42) | | | |
| | Rabbit | Day | Day | Day | Day | Day | Day | Day |
| Group # | # | 0 | 14 | 28 | 42 | 56 | 70 | 84 |
| Study 2 | G3560 | 0 | 994 | 41,300 | 114,000 | 67,600 | 73,100 | 69,800 |
| Immunogen Lot Number 2 | G3561 | 0 | 1,024 | 14,300 | 32,800 | 36,400 | 33,400 | 52,600 |
| Montanide ISA 703 vehicle | G4205 | 0 | 140 | 13,200 | 84,900 | 62,400 | 57,900 | 66,800 |
| GnRH Chimera 2 (TT-3) in | G4206 | 0 | 812 | 16,100 | 33,800 | 47,800 | 81,900 | 110,000 |
| Emulsion = 0.625 mg/ml | G4207 | 0 | 237 | 10,900 | 14,600 | 21,300 | 41,000 | 46,200 |
| GnRH Chimera 3 (TT-2) in | G4208 | 0 | 16,700 | 94,400 | 129,000 | 131,000 | 143,000 | 215,000 |
| Emulsion = 0.625 mg/ml | G4209 | 0 | 0 | 15,200 | 16,900 | 12,900 | 12,400 | 12,200 |
| Peptide Dose = 500 μg/dose | G4210 | 0 | 548 | 34,000 | 55,100 | 74,700 | 127,000 | 152,000 |
| (250 μg/dose of each peptide) | G4211 | 0 | 2,702 | 32,300 | 91,500 | 85,400 | 94,400 | 83,500 |
| NorMDP Dose = 3 μg/dose | G4212 | 0 | 206 | 26,400 | 45,900 | 62,900 | 82,100 | 95,800 |
| Injection Volume = (2 × 0.2) | Mean, Study 2 | 0 | 2,336 | 29,810 | 61,850 | 60,240 | 74,620 | 90,390 |
| ml/injection | Median, Study 2 | 0 | 680 | 21,250 | 50,500 | 62,650 | 77,500 | 76,650 |
| | S.D., Study 2 | 0 | 5,107 | 24,977 | 40,583 | 33,986 | 40,692 | 57,984 |
| Study 1, Group 2 | F6801 | 0 | 219 | 4,179 | 33,900 | 81,500 | 85,300 | 113,000 |
| Immunogen Lot Number1B | G0131 | 0 | 8,659 | 100,000 | 193,000 | 242,000 | 169,000 | 129,000 |
| Montanide ISA 703 vehicle | G0140 | 0 | 305 | 14,800 | 89,500 | 91,300 | 97,800 | 69,000 |
| GnRH Chimera 2 (TT-3) in | G0141 | 0 | 1,071 | 11,000 | 26,600 | 30,600 | 27,500 | 19,300 |
| Emulsion = 0.625 mg/ml | G0142 | 0 | 554 | 16,300 | 64,000 | 32,500 | 31,400 | 31,100 |
| GnRH Chimera 3 (TT-2) in | G0143 | 0 | 1,940 | 32,700 | 86,400 | 70,500 | 65,600 | 68,800 |
| Emulsion = 0.625 mg/ml | Mean, Study 1 | 0 | 2,125 | 29,830 | 82,233 | 91,400 | 79,433 | 71,700 |
| Peptide Dose = 500 μg/dose | Median, Study 1 | 0 | 813 | 15,550 | 75,200 | 76,000 | 75,450 | 68,900 |
| (250 μg/dose of each peptide) | S.D., Study 1 | 0 | 3,263 | 35,647 | 60,172 | 77,950 | 52,134 | 43,356 |
| NorMDP Dose = 3 μg/dose | | | | | | | | |
| Injection Volume = (2 × 0.2) | | | | | | | | |
| ml/iniection | | | | | | | | |

TABLE 7B
Anti-GnRH Antibody Titers for GnRH Chimeric Immunogens Study 3

| | Injection | Injection 1 | Injection 2 | | Injection 3 | | | |
|---|---|---|---|---|---|---|---|---|
| | --> | (Day 0) | (Day 14) | | (Day 42) | | | |
| | Rabbit | Day | Day | Day | Day | Day | Day | Day |
| Group # | # | 0 | 14 | 28 | 42 | 56 | 70 | 84 |
| 1 | H4675 | 0 | 0 | 1,560 | 4,046 | 5,430 | 6,288 | 6,331 |
| Immunogen Lot Number 3A | H4676 | 0 | 0 | 1,054 | 7,182 | 15,600 | 19,700 | 12,600 |
| Montanide ISA 703 vehicle | H4677 | 0 | 0 | 187 | 1,970 | 3,098 | 4,603 | 6,347 |
| GnRH Chimera 13 (TT-10) | H4678 | 0 | 0 | 650 | 2,801 | 2,301 | 2,973 | 7,128 |
| in Emulsion = 2.5 mg/ml | H4679 | 0 | 240 | 555 | 2,991 | 3,383 | 3,094 | 6,338 |
| Peptide Dose = 500 μg/dose | Mean, Group 1 | 0 | 48 | 801 | 3,798 | 5,962 | 7,332 | 7,749 |
| norMDP Dose = 3 μg/dose | Median, Group 1 | 0 | 0 | 650 | 2,991 | 3,383 | 4,603 | 6,347 |
| Injection Volume = | S.D., Group 1 | 0 | 107 | 525 | 2,031 | 5,510 | 7,044 | 2,733 |
| 0.2 ml/injection | | | | | | | | |
| 2 | H4680 | 0 | 286 | 10,700 | 33,600 | 33,500 | 18,600 | 34,300 |
| Immunogen Lot Number 3B | H4681 | 0 | 0 | 2,042 | 9,056 | 10,900 | 7,268 | 15,500 |
| Montanide ISA 703 vehicle | H4682 | 0 | 557 | 7,082 | 30,400 | 30,700 | 18,200 | 32,800 |
| GnRH Chimera 14 (TT-11) in | H4683 | 0 | 192 | 7,865 | 18,800 | 15,400 | 18,000 | 23,000 |
| Emulsion = 2.5 mg/ml | H4684 | 0 | 1,097 | 6,368 | 15,700 | 18,100 | 14,800 | 17,800 |
| Peptide Dose = 500 μg/dose | Mean, Group 2 | 0 | 426 | 6,811 | 21,511 | 21,720 | 15,374 | 24,680 |
| norMDP Dose = 3 μg/dose | Median, Group 2 | 0 | 286 | 7,082 | 18,800 | 18,100 | 18,000 | 23,000 |
| Injection Volume = | S.D., Group 2 | 0 | 425 | 3,132 | 10,264 | 9,868 | 4,778 | 8,557 |
| 0.2 ml/injection | | | | | | | | |
| 3 | H4685 | 0 | 1,577 | 9,622 | 18,800 | 24,200 | 14,900 | 21,100 |
| Immunogen Lot Number 3C | H4686 | 0 | 155 | 10,500 | 16,900 | 12,500 | 7,528 | 13,300 |
| Montanide ISA 703 vehicle | H4687 | 0 | 0 | 5,335 | 7,076 | 5,441 | 3,288 | 6,480 |
| GnRH Chimera 15 (DT-2) in | H4688 | 0 | 0 | 2,405 | 3,163 | 4,126 | 4,519 | 6,449 |
| Emulsion = 1.25 mg/ml | H4689 | 0 | 1,090 | 23,900 | 26,000 | 19,100 | 11,700 | 15,600 |
| Peptide Dose = 500 μg/dose | Mean, Group 3 | 0 | 564 | 10,352 | 14,388 | 13,073 | 8,387 | 12,586 |
| norMDP Dose = 3 μg/dose | Median, Group 3 | 0 | 155 | 9,622 | 16,900 | 12,500 | 7,528 | 13,300 |
| Injection Volume = 2 × 0.2 | S.D., Group 3 | 0 | 726 | 8,253 | 9,221 | 8,642 | 4,876 | 6,266 |
| ml/injection | | | | | | | | |
| 4 | H4690 | 0 | 0 | 0 | 0 | 0 | 0 | 0 |
| Immunogen Lot Number 3D | H4691 | 0 | 0 | 0 | 0 | 538 | 976 | 1,126 |
| Montanide ISA 703 vehicle | H4692 | 0 | 0 | 435 | 416 | 1,444 | 1,179 | 1,526 |
| GnRH Chimera 18 (TT-5) in | H4693 | 0 | 0 | 489 | 2,414 | 2,594 | 2,411 | 4,059 |
| Emulsion = 2.5 mg/ml | H4694 | 0 | 0 | 726 | 3,391 | 4,661 | 3,365 | 6,440 |
| Peptide Dose = 500 μg/dose | Mean, Group 4 | 0 | 0 | 330 | 1,244 | 1,847 | 1,586 | 2,630 |
| norMDP Dose = 3 μg/dose | Median, Group 4 | 0 | 0 | 435 | 416 | 1,444 | 1,179 | 1,526 |
| Injection Volume = 0.2 | S.D., Group 4 | 0 | 0 | 321 | 1,562 | 1,855 | 1,313 | 2,596 |
| ml/injection | | | | | | | | |
| 5 | H4695 | 0 | 0 | 10,600 | 30,700 | 22,800 | 25,000 | 38,500 |
| Immunogen Lot Number 3E | H6175 | 0 | 0 | 2,635 | 10,200 | 7,288 | 17,000 | 27,100 |
| Montanide ISA 703 vehicle | H6176 | 0 | 0 | 493 | 2,531 | 1,302 | 1,984 | 3,115 |
| GnRH Chimera 2 (TT-3) in | H6177 | 0 | 946 | 15,100 | 29,200 | 12,400 | 19,400 | 25,700 |
| Emulsion = 1.5 mg/ml | H6178 | 0 | 1,380 | 24,900 | 24,100 | 12,400 | 11,600 | 7,064 |
| GnRH Chimera 3 (TT-2) in | Mean, Group 5 | 0 | 465 | 10,746 | 19,346 | 11,238 | 14,997 | 20,296 |
| Emulsion = 1.5 mg/ml | Median, Group 5 | 0 | 0 | 10,600 | 24,100 | 12,400 | 17,000 | 25,700 |
| Peptide Dose = 600 μg/dose | S.D., Group 5 | 0 | 655 | 9,877 | 12,400 | 7,917 | 8,723 | 14,808 |
| total (300 μg each) | | | | | | | | |
| norMDP Dose = 3 μg/dose | | | | | | | | |
| Injection Volume = | | | | | | | | |
| 0.2 ml/injection | | | | | | | | |
| 6 | H6179 | 0 | 1,035 | 5,783 | 14,100 | 13,000 | 16,900 | 17,400 |
| Immunogen Lot Number 3F | H6180 | 0 | 0 | 2,511 | 5,357 | 9,357 | 7,336 | 8,055 |
| Montanide ISA 703 vehicle | H6181 | 0 | 0 | 1,300 | 2,780 | 2,749 | 7,611 | 10,200 |
| GnRH Chimera 2 (TT-3) in | H6182 | 0 | 111 | 3,460 | 11,200 | 7,845 | 16,500 | 17,600 |
| Emulsion = 1 mg/ml | H6183 | 0 | 506 | 6,275 | 15,100 | 7,627 | 14,100 | 28,400 |
| GnRH Chimera 3 (TT-2) in | Mean, Group 6 | 0 | 330 | 3,866 | 9,707 | 8,116 | 12,489 | 16,331 |
| Emulsion = 1 mg/ml | Median, Group 6 | 0 | 111 | 3,460 | 11,200 | 7,845 | 14,100 | 17,400 |
| GnRH Chimera 13 (TT-10) in | S.D., Group 6 | 0 | 445 | 2,125 | 5,420 | 3,692 | 4,703 | 7,976 |
| Emulsion = 1 mg/ml | | | | | | | | |
| Peptide Dose = 600 μg/dose | | | | | | | | |
| total (200 μg each) | | | | | | | | |
| norMDP Dose = 3 μg/dose | | | | | | | | |
| Injection Volume = | | | | | | | | |
| 0.2 ml/injection | | | | | | | | |
| 7 | H6184 | 0 | 452 | 11,900 | 49,900 | 42,500 | 35,300 | 142,000 |
| Immunogen Lot Number 3G | H6185 | 0 | 606 | 13,600 | 47,400 | 23,700 | 23,400 | 75,600 |
| Montanide ISA 703 vehicle | H6186 | 0 | 2,414 | 21,700 | 64,600 | 47,800 | 57,400 | 39,600 |
| GnRH Chimera 2 (TT-3) in | H6187 | 0 | 1,050 | 8,551 | 68,800 | 51,900 | 61,200 | 59,500 |
| Emulsion = 1 mg/ml | H6188 | 0 | 746 | 16,800 | 38,200 | 36,300 | 48,700 | 104,000 |
| GnRH Chimera 3 (TT-2) in | Mean, Group 7 | 0 | 1,054 | 14,510 | 53,780 | 40,440 | 45,200 | 84,140 |
| Emulsion = 1 mg/ml | Median, Group 7 | 0 | 746 | 13,600 | 49,900 | 42,500 | 48,700 | 75,600 |
| GnRH Chimera 14 (TT-11) in | S.D., Group 7 | 0 | 792 | 5,002 | 12,661 | 11,035 | 15,735 | 40,019 |
| Emulsion = 1 mg/ml | | | | | | | | |
| Peptide Dose = 600 μg/dose | | | | | | | | |
| total (200 μg each) | | | | | | | | |
| norMDP Dose = 3 μg/dose | | | | | | | | |
| Injection Volume = | | | | | | | | |
| 0.2 ml/injection | | | | | | | | |
| 8 | H6189 | 0 | 4,854 | 52,900 | 125,000 | 69,800 | 99,800 | 97,200 |
| Immunogen Lot Number 3H | H6190 | 0 | 417 | 24,700 | 91,100 | 45,600 | 79,000 | 91,000 |
| Montanide ISA 703 vehicle | H6191 | 0 | 414 | 20,400 | 39,600 | 18,100 | 24,500 | 44,500 |
| GnRH Chimera 2 (TT-3) in | H6192 | 0 | 1,532 | 23,800 | 73,700 | 45,600 | 63,600 | 91,800 |
| Emulsion = 0.5 mg/ml | H6193 | 0 | 326 | 30,000 | 71,300 | 34,900 | 74,200 | 171,000 |
| GnRH Chimera 3 (TT-2) In | Mean, Group 8 | 0 | 1,509 | 30,360 | 80,140 | 42,800 | 68,220 | 99,100 |
| Emulsion = 0.5 mg/ml | Median, Group 8 | 0 | 417 | 24,700 | 73,700 | 45,600 | 74,200 | 91,800 |
| GnRH Chimera 15 (DT-2) in | S.D., Group 8 | 0 | 1,935 | 13,062 | 31,208 | 18,823 | 27,758 | 45,479 |
| Emulsion = 0.5 mg/ml | | | | | | | | |
| Peptide Dose = 600 μg/dose | | | | | | | | |
| total (200 μg each) | | | | | | | | |
| norMDP Dose = 3 μg/dose | | | | | | | | |
| Injection Volume = | | | | | | | | |
| (2 × 0.2) ml/injection | | | | | | | | |
| 9 | H6194 | 0 | 178 | 8,243 | 26,100 | 16,800 | 22,600 | 17,200 |
| Immunogen Lot Number 3I | H6195 | 0 | 1,924 | 21,400 | 56,600 | 39,100 | 66,500 | 65,000 |
| Montanide ISA 703 vehicle | H6196 | 0 | 0 | 2,630 | 4,560 | 4,995 | 5,947 | 3,986 |
| GnRH Chimera 2 (TT-3) in | H6197 | 0 | 0 | 9,295 | 17,100 | 28,400 | 40,400 | 49,600 |
| Emulsion = 1 mg/ml | H6198 | 0 | 0 | 12,200 | 33,900 | 35,600 | 55,200 | 66,100 |
| GnRH Chimera 3 (TT-2) in | Mean, Group 9 | 0 | 420 | 10,754 | 27,652 | 24,979 | 38,129 | 40,377 |
| Emulsion = 1 mg/ml | Median, Group 9 | 0 | 0 | 9,295 | 26,100 | 28,400 | 40,400 | 49,600 |
| GnRH Chimera 18 (TT-5) in | S.D., Group 9 | 0 | 844 | 6,890 | 19,519 | 14,055 | 24,384 | 28,348 |
| Emulsion = 1 mg/ml | | | | | | | | |
| Peptide Dose = 600 μg/dose | | | | | | | | |
| total (200 μg each) | | | | | | | | |
| norMDP Dose = 3 μg/dose | | | | | | | | |
| Injection Volume = | | | | | | | | |
| 0.2 ml/injection | | | | | | | | |
| 10 | H6199 | 0 | 8,813 | 76,600 | 142,000 | 134,000 | 162,000 | 167,000 |
| Immunogen Lot Number 3J | H6200 | 0 | 7,140 | 39,200 | 74,900 | 36,200 | 47,800 | 64,400 |
| Montanide ISA 703 vehicle | H6201 | 0 | 4,507 | 15,700 | 37,000 | 23,300 | 23,600 | 40,400 |
| GnRH Chimera 2 (TT-3) in | H6202 | 0 | 2,270 | 27,400 | 34,700 | 35,800 | 51,800 | 57,200 |
| Emulsion = 0.25 mg/ml | H6203 | 0 | 566 | 16,200 | 34,600 | 30,200 | 39,000 | 68,500 |
| GnRH Chimera 3 (TT-2) in | Mean, Group 10 | 0 | 4,659 | 35,020 | 64,640 | 51,900 | 64,840 | 79,500 |
| Emulsion = 0.25 mg/ml | Median, Group 10 | 0 | 4,507 | 27,400 | 37,000 | 35,800 | 47,800 | 64,400 |
| GnRH Chimera 13 (TT-10) in | S.D., Group 10 | 0 | 3,387 | 25,159 | 46,510 | 46,192 | 55,383 | 50,078 |
| Emulsion = 0.25 mg/ml | | | | | | | | |
| GnRH Chimera 14 (TT-11) in | | | | | | | | |
| Emulsion = 0.25 mg/ml | | | | | | | | |
| GnRH Chimera 15 (DT-2) in | | | | | | | | |
| Emulsion = 0.25 mg/ml | | | | | | | | |
| GnRH Chimera 18 (TT-5) in | | | | | | | | |
| Emulsion = 0.25 mg/ml | | | | | | | | |
| Peptide Dose = 600 μg/dose | | | | | | | | |
| total (100 μg each) | | | | | | | | |
| norMDP Dose = 3 μg/dose | | | | | | | | |
| Injection Volume = | | | | | | | | |
| (2 × 0.2) ml/injection | | | | | | | | |

Study 2 was undertaken to verify the high titers obtained with the mixture of chimeric immunogens (2+3) in study 1 (FIG. 3). Equivalent titers were obtained for this mixture of chimeric immunogens in both the studies. Of all the chimeric immunogen combinations tested prior to study 1, the mixture of chimeric immunogens (2+3) was the most immunogenic, and was therefore used as a control in study 3.

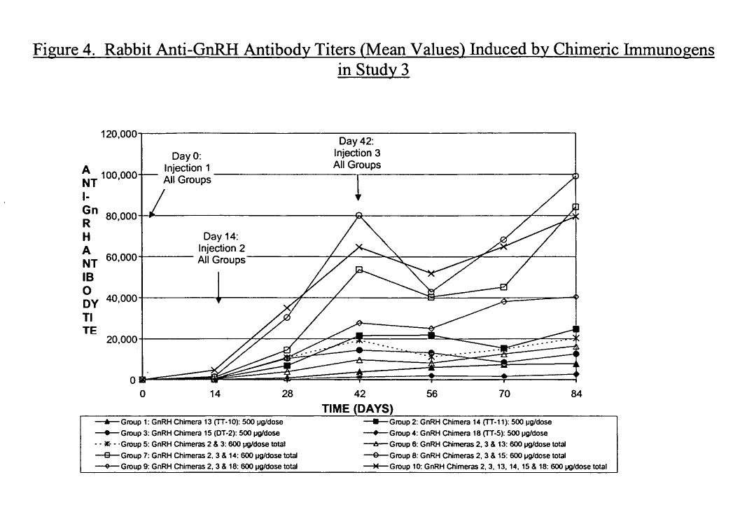

Study 3 assessed four new chimeric immunogens, numbers 13, 14, 15 and 18, alone and formulated in selected combinations. Chimeric immunogens 2 and 3 were included in the study. All rabbits responded to each of the chimeric immunogens with the production of anti-GnRH antibody titers. The various formulations differed in their capacity to elicit anti-GnRH antibody responses. By themselves, chimeric immunogens 13 and 18 were weak immunogens (FIG. 4); whereas, chimeric immunogens 14 and 15 induced stronger responses. The anti-GnRH antibody levels induced by chimeric immunogen 14 were comparable to levels historically raised by the D17DT Immunogen (a conjugate of GnRH linked to DT). When tested in combination with chimeric immunogen (2+3), chimeric immunogen 13 reduced the antibody titer in comparison with immunogen formulated with chimeric immunogens (2+3) only (FIG. 4); whereas, the addition of chimeric immunogen 18 to (2+3) resulted in a formulation of comparable immunogenicity to chimeric immunogens (2+3). Notably, the addition of chimeric immunogen 14 and (particularly) 15 to chimeric immunogens (2+3) significantly enhanced anti-GnRH titers (Table 8, Student's t-Test, p≦0.05). A mixture of all 6 chimeric immunogens was also strongly immunogenic and statistically significant from Study 3/Group 5 (chimeric immunogens (2+3)). Thus, certain combinations of GnRH chimeric immunogens are particularly potent anti-GnRH immunogens, while other combinations depress immunogenicity.

TABLE 8
Statistical Comparisons of Peak Antibody Titers (Student's
t-Test; Equal Variances, Two-Tail, p ≦ 0.05)

| | | | | Signif- |
|---|---|---|---|---|
| | | | T-test | icant? |
| | Groups Compared | | p value | (p ≦ 0.05) |
| Study/Group | Study 3/Group 8 | Study 3/Group 10 | 0.54 | NO |
| Day | 84 | 84 | | |
| Mean Titer | 99,100 | 79,500 | | |
| Study/Group | Study 3/Group 7 | Study 3/Group 10 | 0.88 | NO |
| Day | 84 | 84 | | |
| Mean Titer | 84,140 | 79,500 | | |
| Study/Group | Study 3/Group 5 | Study 3/Group 7 | 0.01 | YES |
| Day | 84 | 84 | | |
| Mean Titer | 20,296 | 84,140 | | |
| Study/Group | Study 3/Group 5 | Study 3/Group 8 | 0.01 | YES |
| Day | 84 | 84 | | |
| Mean Titer | 20,296 | 99,100 | | |
| Study/Group | Study 3/Group 5 | Study 3/Group 9 | 0.20 | NO |
| Day | 84 | 84 | | |
| Mean Titer | 20,296 | 40,377 | | |
| Study/Group | Study 3/Group 5 | Study 3/Group 10 | 0.03 | YES |
| Day | 84 | 84 | | |
| Mean Titer | 20,296 | 79,500 | | |
| Study/Group | Study 1/Group 1 | Study 1/Group 3 | 0.38 | NO |
| Day | 70 | 42 | | |
| Mean Titer | 70,200 | 43,449 | | |
| Study/Group | Study 1/Group 2 | Study 1/Group 3 | 0.75 | NO |
| Day | 42 | 42 | | |
| Mean Titer | 52,040 | 43,449 | | |

Injection site reactions were assessed in these studies. The individual injection site reaction scores are given in Tables 9 A/B/C. The mean scores are given in Table 9D. Assessment of injection site reactions indicated that the chimeric immunogens were generally well tolerated, with the exception of chimeric immunogen 14, which was strongly reactogenic by itself and in combination with chimeric immunogens (2+3), and in the mixture of all six peptides. This contrasted with chimeric immunogen 15, which was immunogenic when administered singly but significantly less reactogenic than chimeric immunogen 14. These striking differences in reactogenicity were unexpected.

Table 9. Gross Pathology Observations of Injection Site Reactions on Day 84

TABLE 9A
Individual Rabbit Scores in Study 2

| | | REACTION | | | | | | |
|---|---|---|---|---|---|---|---|---|
| STUDY/GROUP | RABBIT | Injection 1 | | Injection 2 | | Injection 3 | | |
| NUMBER | NUMBER | SITE 1 | SITE 2 | SITE 1 | SITE 2 | SITE 1 | SITE 2 | BIOPSY |
| 2 | G356 | 0.5 | 0.5 | 0.5 | 0.5 | 1 | 1.5 | X |
| | G356 | 0.5 | 0.5 | 2 | 2 | 1 | 1 | X |
| | G420 | 0.5 | 0.5 | 0.5 | 0 | 1 | 1.5 | |
| | G420 | 0.5 | 1 | 0.5 | 0.5 | 1 | 3 | |
| | G420 | 0.5 | 0.5 | 1 | 1 | 1 | 1 | |
| | G420 | 0.5 | 0 | 1 | 0.5 | 0.5 | 1 | |
| | G420 | 0.5 | 0.5 | 0 | 0.5 | 0.5 | 0.5 | |
| | G421 | 0.5 | 0.5 | 0.5 | 0.5 | 1 | 1 | |
| | G421 | 0 | 0.5 | 0.5 | 1 | 1 | 1 | |
| | G421 | 0.5 | 0.5 | 0.5 | 0.5 | 2 | 1 | |

TABLE 9B
Individual Rabbit Scores in Study 3

| | | REACTION SCORES | | | | | | |
|---|---|---|---|---|---|---|---|---|
| GROUP | RABBIT | Injection 1 | | Injection 2 | | Injection 3 | | |
| NUMBER | NUMBER | SITE 1 | SITE 2 | SITE 1 | SITE 2 | SITE 1 | SITE 2 | BIOPSY |
| Group 1 | H4675 | 0 | | 0.5 | | 1.5 | | X |
| | H4676 | 0 | | 0.5 | | 1 | | X |
| | H4677 | 0 | | 0.5 | | 1 | | |
| | H4678 | 0.5 | | 1 | | 1 | | |
| | H4679 | 0.5 | | 1 | | 1 | | |
| Group 2 | H4680 | 0.5 | | 1 | | 3 | | X |
| | H4681 | 0.5 | | 0.5 | | 1 | | X |
| | H4682 | 0.5 | | 2 | | 2 | | |
| | H4683 | 0.5 | | 1 | | 1.5 | | |
| | H4684 | 0 | | 0.5 | | 1 | | |
| Group 3 | H4685 | 0.5 | 0 | 0 | 0.5 | 0.5 | 0 | X |
| | H4686 | 0 | 0 | 0.5 | 0 | 0 | 0 | X |
| | H4687 | 0 | 0 | 0 | 0 | 0 | 0 | |
| | H4688 | 0.5 | 0 | 0 | 0 | 0 | 0 | |
| | H4689 | 0.5 | 0.5 | 0 | 0 | 0.5 | 0 | |
| Group 4 | H4690 | 0 | | 0 | | 0 | | X |
| | H4691 | 0 | | 0 | | 0.5 | | X |
| | H4692 | 0.5 | | 0.5 | | 0.5 | | |
| | H4693 | 0.5 | | 0.5 | | 0.5 | | |
| | H4694 | 0.5 | | 0.5 | | 1 | | |
| Group 5 | H4695 | 0 | | 1 | | 3 | | X |
| | H6175 | 0 | | 0.5 | | 0.5 | | X |
| | H6176 | 0 | | 0.5 | | 0.5 | | |
| | H6177 | 0.5 | | 0.5 | | 0.5 | | |
| | H6178 | 0 | | 0.5 | | 0.5 | | |
| Group 6 | H6179 | 0 | | 0.5 | | 1 | | X |
| | H6180 | 0 | | 0.5 | | 0.5 | | X |
| | H6181 | 0.5 | | 0.5 | | 1 | | |
| | H6182 | 0.5 | | 0 | | 0.5 | | |
| | H6183 | 0.5 | | 0.5 | | 1 | | |
| Group 7 | H6184 | 0.5 | | 1 | | 3 | | X |
| | H6185 | 0.5 | | 1 | | 2 | | X |
| | H6186 | 0.5 | | 0.5 | | 0.5 | | |
| | H6187 | 0.5 | | 1 | | 3 | | |
| | H6188 | 0.5 | | 1 | | 2 | | |
| Group 8 | H6189 | 0.5 | 0 | 1 | 1 | 1 | 0.5 | X |
| | H6190 | 0 | 0 | 0.5 | 0.5 | 1 | 1 | X |
| | H6191 | 0 | 0 | 0.5 | 0.5 | 1 | 0.5 | |
| | H6192 | 0.5 | 0.5 | 1 | 1 | 1 | 0.5 | |
| | H6193 | 0 | 0.5 | 0.5 | 0.5 | 1 | 0.5 | |
| Group 9 | H6194 | 0 | | 0.5 | | 0.5 | | X |
| | H6195 | 0 | | 0.5 | | 1.5 | | X |
| | H6196 | 0 | | 0.5 | | 0.5 | | |
| | H6197 | 0.5 | | 0.5 | | 1 | | |
| | H6198 | 0 | | 0.5 | | 1 | | |
| Group 10 | H6199 | 0.5 | 0.5 | 1 | 1 | 1.5 | 2 | X |
| | H6200 | 0.5 | 0.5 | 0.5 | 0.5 | 2 | 2 | X |
| | H6201 | 0.5 | 0.5 | 1 | 0.5 | 1 | 1 | |
| | H6202 | 0.5 | 0.5 | 1 | 1 | 2 | 2 | |
| | H6203 | 0.5 | 0.5 | 1 | 1 | 2 | 1 | |

TABLE 9C
Mean Injection Site Reaction Scores

| | MEAN REACTION SCORES | | | | | | REACTION SCORES > 1 | | | | | |
|---|---|---|---|---|---|---|---|---|---|---|---|---|
| | Injection 1 | | Injection 2 | | Injection 3 | | Injection 1 | | Injection 2 | | Injection 3 | |
| TREATMENT | SITE 1 | SITE 2 | SITE 1 | SITE 2 | SITE 1 | SITE 2 | SITE 1 | SITE 2 | SITE 1 | SITE 2 | SITE 1 | SITE 2 |
| Study A: 500 μg (total) | 0.5 | 0.5 | 0.7 | 0.7 | 1 | 1.3 | 0 | 0 | 1 | 1 | 1 | 3 |
| of GnRH Chimeras (TT-2) + | | | | | | | | | | | | |
| (TT-3) + 3 μg norMDP | | | | | | | | | | | | |
| Study B Gp 1: Chimera 13 | 0.2 | | 0.7 | | 1.1 | | 0 | | 0 | | 1 | |
| (TT-10) | | | | | | | | | | | | |
| Study B Gp 2: Chimera 14 | 0.4 | | 1 | | 1.7 | | 0 | | 1 | | 3 | |
| (TT-11) | | | | | | | | | | | | |
| Study B Gp 3: Chimera 15 | 0.3 | 0.1 | 0.1 | 0.1 | 0.2 | 0 | 0 | 0 | 0 | 0 | 0 | 0 |
| (DT-2) | | | | | | | | | | | | |
| Study B Gp 4: Chimera 18 | 0.3 | | 0.3 | | 0.5 | | 0 | | 0 | | 0 | |
| (TT-5) | | | | | | | | | | | | |
| Study B Gp 5: Chimeras 2 | 0.1 | | 0.6 | | 1 | | 0 | | 0 | | 1 | |
| (TT-3) & 3 (TT-2) | | | | | | | | | | | | |
| STudy B Gp 6: Chimeras 2 | 0.3 | | 0.4 | | 0.8 | | 0 | | 0 | | 0 | |
| (TT-3) 3 (TT-2) & 13 (TT-10) | | | | | | | | | | | | |
| Study B Gp 7: Chimeras 2 | 0.5 | | 0.9 | | 2.1 | | 0 | | 0 | | 4 | |
| (TT-3), 3 (TT-2) & 14 (TT-11) | | | | | | | | | | | | |
| Study B Gp 8: Chimeras 2 | 0.2 | 0.2 | 0.7 | 0.7 | 1 | 0.6 | 0 | 0 | 0 | 0 | 0 | 0 |
| (TT-3), 3 (TT-2) & 15 (DT-2) | | | | | | | | | | | | |
| Study B Gp 9: Chimeras 2 | 0.1 | | 0.5 | | 0.9 | | 0 | | 0 | | 1 | |
| (TT-3) 3 (TT-2) & 18 (TT-5) | | | | | | | | | | | | |
| Study B Gp 10: Chimeras 2 | 0.5 | 0.5 | 0.9 | 0.8 | 1.7 | 1.6 | 0 | 0 | 0 | 0 | 4 | 3 |
| (TT-3), 3 (TT-2), 13 (TT-10), | | | | | | | | | | | | |
| 134 (TT-11), 15 (DT-2) & | | | | | | | | | | | | |
| 18 (TT-5) | | | | | | | | | | | | |

In studies 3 and 4 (Group 1), where chimeric immunogens (2+3) were prepared identically, the reaction scores of groups receiving the different preparations were equivalent. Groups 2 and 3 in study 4, where chimeric immunogens (2+3) were delivered in 0.2 ml dose volumes, low injection site reactions were seen. Thus, these immunogen preparations were well tolerated by the rabbits, despite solubility differences observed in their preparations.

In general, the histologic readings of the injection site biopsies were in accord with the gross evaluations (Tables 10A and 10B). It should be noted that the visual and microscopic numerical scoring systems are not calibrated against one another, and that microscopic pathology is frequently observed in the absence of visual injection site reactions. Lower reaction scores were associated with more recent injections, as the inflammatory reactions at earlier sites had more time to resolve.

Table 10. Histopathological Evaluation of Injection Sites on Day 84

TABLE 10A
Histopathological Scores of Injection Sites in Study 2

| | | HISTOPATHOLOGY SCORES | | | | | |
|---|---|---|---|---|---|---|---|
| STUDY/GROUP | RABBIT | Injection 1 | | Injection 2 | | Injection 3 | |
| NUMBER | NUMBER | SITE 1 | SITE 2 | SITE 1 | SITE 2 | SITE 1 | SITE 2 |
| 2 | G3560 | 2 | 2 | 2.5 | 2 | 2.5 | 3 |
| | G3561 | 2 | 2 | 3 | 3 | 3 | 3 |

TABLE 10B
Histopathological Scores of Injection Sites in Study 3

| | HISTOPATHOLOGY SCORES | | | | | |
|---|---|---|---|---|---|---|
| RABBIT | Injecion 1 | | Injection 2 | | Injection 3 | |
| NUMBER | SITE 1 | SITE 2 | SITE 1 | SITE 2 | SITE 1 | SITE 2 |
| H4675 | 0.5 | | 2.5 | | 3 | |
| H4676 | 0.5 | | 1 | | 2 | |
| H4680 | 1 | | 1.5 | | 3 | |
| H4681 | 1 | | 3 | | 3 | |
| H4685 | 0.5 | 1 | 1 | 0.5 | 1 | 1.5 |
| H4686 | 1 | 1 | 1 | 1.5 | 0 | 0 |
| H4690 | 0 | | 0.5 | | 0.5 | |
| H4691 | 0.5 | | 1 | | 3 | |
| H4695 | 0.5 | | 3 | | 3 | |
| H6175 | 0.5 | | 2.5 | | 3 | |
| H6179 | 0.5 | | 2 | | 2.5 | |
| H6180 | 0.5 | | 0.5 | | 1 | |
| H6184 | 1 | | 2.5 | | 3 | |
| H6185 | 2 | | 3 | | 3 | |
| H6189 | 2 | 1 | 2.5 | 0.5 | 1.5 | 2 |
| H6190 | 1 | 1 | 1 | 0.5 | 2 | 2.5 |
| H6194 | 1 | | 1.5 | | 2 | |
| H6195 | 0.5 | | 2 | | 2.5 | |
| H6199 | 2 | 2.5 | 1 | 2.5 | 3 | 2 |
| H6200 | 2.5 | 1.5 | 3 | 3 | 2.5 | 3 |

These results show that some, but not all, combinations of these chimeric immunogens are potent immunogens against GnRH. The addition of GnRH chimeric immunogens 14 and 15 to a mixture of chimeric immunogens (2+3) enhances immunogenicity. However, some chimeric immunogens are found to suppress immugenicity and or increase reactigenicity. One of ordinary skill in the art will readily appreciate how to determine whether particular chimeric immunogens cause synergistic enhancement of immunogens or suppression according to the methods taught in the present specification. The immunogenicity/reactogenicity profiles of individual or combination chimeric immunogens cannot be predicted, and must be determined empirically. In the studies of Example III, the optimal immunogen was found to be comprised of a mixture of chimeric immunogens (2+3+15).

The specifications of each of the U.S. Patents and the texts of each of the references cited in the present specification are here incorporated by reference in their entireties.

While this invention has been described with an emphasis upon preferred embodiments, it will be clear and obvious to those of ordinary skill in the art that variations of the preferred embodiments of the invention may be advantageously practiced. Accordingly, the present invention as contemplated includes all modifications encompassed within the spirit and scope of the specification as defined by the following claims.

1. A composition comprising:
a) a first chimeric immunogen comprising
i) a helper T-lymphocyte epitope, fused through
ii) a spacer moiety, to
iii) an immunomimic of a target antigen; and
b) a second chimeric immunogen comprising
i) a different helper T-lymphocyte epitope, fused through
ii) a spacer moiety, to
iii) the immunomimic of the target antigen
wherein the helper T-lymphocyte epitopes synergistically enhance or suppress the antibody response to the target antigen.

2. The composition of claim 1, wherein the helper T-lymphocyte epitopes synergistically enhance the antibody response to the target antigen.

3. The composition of claim 1, wherein the helper T-lymphocyte epitopes suppress the antibody response to the target antigen.

4. The composition of claim 1, wherein the target antigen is a peptide.

5. The composition of claim 4, wherein the target antigen peptide is a peptide hormone.

6. The composition of claim 5, wherein the peptide hormone is a mammalian peptide hormone.

7. The composition of claim 6, wherein the mammalian peptide hormone is GnRH.

8. The composition of claim 7, wherein at least one of the immunomimics of the target antigen comprises between about 5 and about 10 contiguous amino acids of the amino acid sequence of GnRH (SEQ ID NO: 1).

9. The composition of claim 7, wherein at least one of the GnRH-immunomimics has an acetylated amino-terminal glutamic acid or an amidated carboxy-terminal glycine.

10. The composition according to claim 7, wherein the combination of helper T-lymphocyte epitopes has a synergistic effect on the antibody response to GnRH, and wherein at least one of the chimeric immunogens is selected from the group consisting of the peptides defined by SEQ ID NO: 10, SEQ ID NO: 11, SEQ ID NO: [Chimera 12], SEQ ID NO: [Chimera 13], SEQ ID NO: [Chimera 15], and SEQ ID NO: [Chimera 18].

11. The composition of claim 10, comprising the immunogens defined by the peptides of SEQ ID NO: 10, SEQ ID NO: 11, and SEQ ID NO: [Chimera 13].

12. The composition of claim 10, comprising the immunogens defined by the peptides of SEQ ID NO: 10, SEQ ID NO: 11, and SEQ ID NO: [Chimera 15].

13. The composition of claim 1, wherein at least one of the helper T-lymphocyte epitopes is not contained within the target antigen.

14. The composition of claim 13, wherein two or more of the helper T-lymphocyte epitopes are not contained within the target antigen.

15. The composition of claim 1, wherein at least one of the spacer moieties comprises a linkage selected from the group consisting of —O—R—CO—, —NH—R—CO—, —NH—R—NH—, —O—R—NH— or —NH—R—CH₂—, in which R is a saturated or unsaturated hydrocarbon chain optionally substituted and/or interrupted by one or more aromatic radicals or by hereroatoms selected from N, O, or S.

16. The composition of claim 1, wherein at least one of the spacer moieties is a peptide.

17. The composition of claim 16, wherein at least one of the spacer moiety peptides is selected from the group consisting of a hinge peptide, Gly-Gly (SEQ ID NO:), Gly-Pro-Ser-Leu (SEQ ID NO: 5), Ser-Ser-Gly-Pro-Ser-Leu (SEQ ID NO: 6), and Ser-Ser-Gly-Pro-Ser-Leu-Lys-Leu (SEQ ID NO: 7).

18. The composition of claim 4, wherein the immunomimic is a peptide, and wherein at least one of the helper T-lymphocyte epitopes is fused through a spacer moiety to the amino-terminus or the carboxy-terminus of the immunomimic peptide.

19. The composition of claim 1, wherein the immunomimic is a peptide, and wherein at least one of the helper T-lymphocyte epitopes is fused through a spacer peptide at a position other than the amino-terminus or the carboxy-terminus of the immunomimic peptide.

20. The composition of claim 1, wherein at least one of the helper T-lymphocyte epitopes is fused to the spacer moiety through a non-peptide bond.

21. The composition of claim 1, wherein at least one of the spacer moieties is fused to the immunomimic peptide through a non-peptide bond.

22. The composition of claim 1, wherein at least one of the immunogens is a synthetic immunogen.

23. The composition of claim 1, wherein at least one of the immunogens is a recombinant immunogen.

24. A method of eliciting an antibody response to a target antigen in a patient, comprising:
administering to the patient a composition comprising:
a) a first chimeric immunogen comprising
i) a helper T-lymphocyte epitope, fused through
ii) a spacer moiety, to
iii) an immunomimic of the target antigen, and
b) a second chimeric immunogen comprising
i) a different helper T-lymphocyte epitope, fused through
ii) a spacer moiety, to
iii) an immunomimic of the target antigen;
wherein the helper T-lymphocyte epitopes synergistically enhance the antibody response to the target antigen.

25. A method of suppressing an antibody response to a target antigen in a patient, comprising, administering to the patient a composition comprising:
a chimeric immunogen comprising
i) a helper T-lymphocyte epitope that is not found in the target antigen, fused through
ii) a spacer moiety, to
iii) an immunomimic of the target antigen
wherein the helper T-lymphocyte epitope suppresses the antibody response to the target antigen.

26. The method according to claim 25, wherein the antibody response to the target antigen is an autoimmune antibody response, an allergic antibody response or an antibody response to a graft.

27. A pharmaceutical composition comprising the composition of claim 1, and a pharmaceutically acceptable carrier.
